(19) United States
(12) Patent Application Publication
(10) Pub. No.: US 2011/0038936 A1
(43) Pub. Date: Feb. 17, 2011

(54) SYSTEM AND METHOD FOR ELECTROSPUN DRUG LOADED BIODEGRADABLE CHEMOTHERAPY APPLICATIONS

(76) Inventors: Kimberly Ann Griswold, Bloomfield, NJ (US); Charles J. Prestigiacomo, East Hanover, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/858,435

(22) Filed: Aug. 17, 2010

Related U.S. Application Data
(60) Provisional application No. 61/234,546, filed on Aug. 17, 2009.

(51) Int. Cl. A61K 9/10; A61K 31/175; A61K 31/704; A61K 31/407; A61K 31/513; A61K 31/519; A61K 31/255; A61K 33/24; A61K 31/17; A61K 31/337; A61K 31/437; A61K 31/52; A61P 7/04; A61P 7/00; A61P 35/00

(52) U.S. Cl. 424/486; 424/484; 514/589; 514/34; 514/410; 514/274; 514/249; 514/517; 424/649; 514/588; 514/449; 514/286; 514/263.3

(57) ABSTRACT

Biodegradable resorbable drug delivery systems characterized by an electrospun biodegradable resorbable polymeric fiber matrix with at least one therapeutic agent incorporated into the fibers of the matrix, wherein the fiber matrix has an interfibrillar space of at least 65% by volume. Therapeutic methods for delivering a chemotherapeutic agent to body cavities from which a tumor has been excised and for strengthening weakened blood vessel walls are also disclosed.

### FIELD OF THE INVENTION

This invention relates to the treatment of cancer. More specifically it relates to the treatment of cancer via chemo-therapy implant.

### BACKGROUND OF THE INVENTION

A major area relevant to vascular malformations is the manifestation of malignant tumor masses embedded in neural tissue. Most glial tumors have two growth patterns: solid tumor tissue and infiltrating tumor cells, which are judged by progressive enlargement on a computerized tomography (CT) or magnetic resonance imaging (MRI). Solid tumor tissue is characterized by the increase in mass from the production of new blood vessels from those that supply the brain with oxygen, glucose, and other nutrients. The new blood vessels function to supply the growing tumor cells with oxygen, glucose, and other nutrients, directly infiltrating the source of nutrients that should supply the brain. Low grade (low blood supply needed, anaplastic or malignant require large blood supply) tumors may not be apparent on a CT or MRI due to the lack of contrast enhancement and may be removed without neurological deficit (solid tumor tissue either displaces or replaces brain substance), depending on the lesion's location. (See, for example, http:/www.braintumorfoundation.org.)

Most low-grade infiltrating tumor cells are visible as a hypodensity on a CT and increased T1 and T2 signal on an MRI because the tumor cells tend to draw water into the infiltrated brain tissue. When isolated tumor cells are not great enough in number to change the local osmolality and thus increase the amount of water surrounding them, the CT and MRI will show no changes and appear normal in the area Id. If some tumor cells still reside in the tissue after treatment, it is guaranteed that the tumor will return.

There are various forms of treatment for these malformations. These include: embolization, radiation therapy (officially known as stereotactic radiotherapy), stereotactic brachytherapy, boron neutron capture therapy (BNCT), surgery, and chemotherapy.

The advantages of chemotherapeutic agents include the fact that they are the least invasive and are not constrained by physical parameters. Most agents like BCNU (bis-chloronitrosourea or carmustine), PCNU (1-(2-chloroethyl)-3-(2,6-dioxo-3-piperidyl)-1-nitrosourea), Procarbazine and Carboplatin are incorporated into and alkylate the DNA of rapidly dividing tumor cells. Vincristine poisons the mitotic process and Etoposide (commonly referred to as VP-16), incorporates itself into tumor cell wall proteins and inhibits the production of microtubules. Topotecan enhances the effect of radiation therapy by intravenously affecting rapidly dividing tumor cells and normal cells from bone marrow and bowel. Most of these agents may result in the reduction of white blood cells (causing severe, potentially life threatening infections), red blood cells (causing anemia), and platelets (thrombocytopenia—resulting in blood clotting disorders and bleeding). However, they are the only agents that can overcome the problems with glial primary brain tumors. Many tumor cells infiltrate surrounding brain tissue where the blood-brain barrier (which prevents the passage of large molecules) is intact. Agents can reach cells in the major mass of the tumor via leaky tumor blood vessels, which provide life support for the tumor. The blood-brain barrier protects tumor cells residing within intact brain tissue surrounding the tumor, but can be disrupted medically by chemotherapy.

Eisai Inc. markets polyanhydride Gliadel® wafers for treating a type of brain cancer called glioblastoma multiforme. These wafers were first discovered by Henry Brem as the only drug delivery device to increase survival time in patients with Stage 4 glioblastoma multiforme. Moses, et al., Cancer Cell, 4, 337-341 (2003).

The wafers consist of poly (1,3-bis-(p-carboxyphenoxy propane)-co-(sebacic anhydride)) 20:80 (p(CPP:SA) 20:80) impregnated with carmustine (BCNU), a toxic antitumor medication and are implanted at the tumor site, and release the drug locally where it is needed while reducing systemic side effects such as liver or kidney damage and pulmonary toxicity (particularly harmful to persons under the age of 12). However, Gliadel® does have other side affects because of the means and location of wafer implantation. In clinical trials, 19% of patients reported new or worsened seizure symptoms and 14% experienced healing abnormalities (such as cerebral fluid leaks, subdural fluid collections, and wound breakdown). Westphal, et al., Neuro-Oncology, 79-88 (2003). Other adverse reactions included intracranial infections, pain in back and chest, hypertension, diarrhea, fever, rashes, and urinary tract infections.

A combination of treatments is the best method for curing brain tumors. Treatments can be tailored to the specific tumor type and patient considerations but require additional steps, time and coordination. (See, for example, www.cumc.columbia.edu/dept/cerebro/AVM.html. Because of the complications of all these procedures, i.e., the high probability of lingering cancer cells that may manifest itself into a tumor, the risk of bleeding, and secondary toxic affects, a drug delivery system that avoids such complications is desirable. Non-invasive procedures that deliver chemotherapy to the target site without traveling through the circulatory system, like enteral methods, are the least likely to cause complications.

Electrospinning is an atomization process that applies a high voltage to an ejectable polymer solution. When an external electrostatic field is applied to a conducting fluid (e.g., a polymer solution or a polymer melt), a suspended conical droplet is formed, whereby the surface tension of the droplet is in equilibrium with the electric field. The basic principle behind this process is that an electric voltage sufficient enough to overcome the surface tension of a polymeric solution causes the polymer droplets to elongate so that the polymer is splayed randomly as very fine fibers, which, when collected on a grounded metal plate, form non-woven mats.

The properties of polymeric matrices depend on multiple factors including their chemical structure, degree of polymerization, orientation of chain molecules, crystallinity, pack-age density and cross linking between individual molecules. Variation in these factors generally determine the morphology of the polymeric product. The parameters of voltage, flow rate, needle gauge size, distance to collection plate, and polymer solution concentration during processing are a few that will impact the properties of the fibrous product. In addition, the homogeneity of the polymeric mixtures must be considered to provide optimal texture and release patterns. The prior art does not provide sufficient guidance to enable one of skill in the art of drug delivery and tissue engineering to use a polymer and an active drug in an electrospinning method using routine experimentation, because the variability of each of such parameters impact the properties of the final product.

U.S. Pat. No. 4,323,525 is directed to a process for the production of tubular products by electrostatically spinning a liquid containing a fiber-forming material. The process involves introducing the liquid into an electric field through a nozzle under conditions to produce fibers of the fiber-forming material, which tend to be drawn to a charged collector, and collecting the fibers on a charged tubular collector which rotates about its longitudinal axis, to form the fiberous tubular product. It is also disclosed that several nozzles can be used to increase the rate of fiber production. However, there is no suggestion or teaching of adding a drug or preparing drug delivery systems, and more importantly there is no teachings as to how to control the physical characteristics of the polymeric product, other than by controlling the charge and rotation speed of a collector.

U.S. Pat. No. 4,689,186 is directed to a process for the production of polyurethane tubular products by electrostatically spinning a fiber-forming liquid containing the polyurethane. It is disclosed that auxiliary electrodes can be placed around the collector to help facilitate collection of the fibers. There is no teaching or suggestion of independently controlling jet formation, jet acceleration and fiber collection, or incorporating a drug into the polymeric matrix to prepare a drug delivery system.

Polymeric matrices for drug delivery have been produced by variety of techniques in the art. Electrospinning techniques have also been shown effective for embedding a therapeutic substance in a polymeric matrix for creating micron-scale systems. U.S. Pat. No. 7,712,765 is directed to a drug-containing polymeric membrane wherein the active ingredient is an antibiotic. The system is used to alleviate risk of post surgical infections. However, there is no teaching or suggestion of adding anti-neoplastic agents to a non-woven nanozied polyester matrix.

Current treatments, such as the Gliadel® wafer, are placed within the tissue cavity after excision of the tumor. The placement of the wafers occurs when the patient is lying down. However, during patient recovery when the patient's body changes positions, the wafers do not maintain contact with the cavity walls. Instead these wafers float in the CSF or agglomerate to one side of the cavity. This prevents direct contact and delivery of the active agents to the entire area of the cavity and requires delivery to occur through a liquid medium. In tumor bed locations where there is a continuous flow of CSF, this lack of contact and failure to provide direct delivery of the chemotherapeutic agent decreases the efficiency of the drug delivery device, ultimately decreasing patient survivability.

Furthermore, direct contact with brain tissue allows for greater drug effect on residual tumor cells. Residual tumor cells are those cells which the surgeons generally fail to remove during a surgical resection of a solid tumor. Residual tumor cells frequently cause reappearance of the cancer tissue and ultimately the solid tumor. Treatment of these types of cell is complicated even more under the dynamic conditions and continuous flow of the cerebrospinal fluid (CSF). Maintaining drug contact is a goal of any therapeutic modality to ensure that the entire excised or resected area is exposed to an adequate anti-tumor drug regimen, minimizing the risk of residual tumor cells to infiltrate into other regions of the body. None of the prior art formulations have addressed this need.

### SUMMARY OF THE INVENTION

The shortcomings of prior art are addressed by the present invention. The present invention offers improved tissue adhesion and subsequently improved delivery of active agents as compared to conventional micro-scale treatment modalities. The present invention relates to new self adhering drug delivery systems capable of destroying residual cancer cells post surgical resection of a tumor. In another aspect polymer matrices are provided that adhere to the interior of and strengthen weak areas of blood vessels such as aneurisms.

The present invention incorporates the discovery that modifying process conditions for electrospinning drug-containing polymers produces polymeric fiber matrix drug delivery systems that have different interfibrillar spaces, morphologies, very large or small surface area to volume ratios and pore sizes. All such features contribute to a improved local delivery of a drug of interest at the site of delivery.

According to one aspect of the present invention, a biodegradable resorbable drug delivery system is provided characterized by an electrospun biodegradable resorbable polymeric fiber matrix with at least one therapeutic agent incorporated into the fibers of the fiber matrix, wherein the fiber matrix has an interfibrillar space of at least 65% by volume. Despite the large volume of interfibrillar space, the polymeric fiber matrices of the present invention are semi-permeable and exhibit fluid migration properties akin to membranes or hydrogels.

The volume of interfibrillar space also provides fiber matrices with adhesive properties that can be used to promote coagulation (aneurisms) and attaching of the matrix to wound sites (something Gliadel has issues with). The present invention includes matrices with top and bottom surfaces of two different textures (pressed smooth and irregularly fibrous) that promote adhesion, and adapt surface variations. That is, because of the adhesiveness, if there was a bit of a breakdown—such as a leak, the material is able to seal the leak very quickly, and because of the adhesiveness the material is able to hold approximately 10 to 150 times it's weight, which makes it well suited for blood vessel repair. The present invention therefore also provides a device formed from hydrophobic polymer fibers that can bear significant multiples of it's own weight and yet has adaptable self-adhesive and self-sealing capabilities when attached to living tissues under physiological conditions.

For certain exemplary embodiments of the present invention the drug delivery design will be an device for implantation following surgical excision of a tumor mass. In at least one embodiment, the electrospinning method has been chosen to create nonwoven fibrous polymeric drug delivery compositions that have a drug homogenously dispersed throughout the fiber matrix with an interfibrillar space that facilitates tissue adhesion for drug delivery thereto. The electrospinning method employed in multiple exemplary embodiments of the present invention is the most efficient and controlled method to produce the presently described fibers on a nanoscale level.

In certain exemplary embodiments of the present invention fibrous mats in the shape and size of wafers such as Gliadel® wafers are chosen because they are easy to handle and manipulate when placed inside a tissue cavity where a tumor once resided. Due to the porous nature of the fiber matrix, the presently disclosed electrospun fibers have improved adhesion to the walls of the post-surgical cavities where residual tumor cells may reside.

Accordingly, another aspect of the invention provides a method of delivering a chemotherapy drug to tissues from which a tumor has been excised in a patient by contacting the tissues with the drug delivery system of the present invention containing an effective amount of a chemotherapeutic agent for delivery to the tissues.

In one exemplary embodiment, the present invention employs DL-polylactic-glycolic-acid (DL-PLGA) as the polymer of choice to provide steady drug delivery in direct contact to the tissue cavity continuously for a period of at least two months. By directly implanting the presently disclosed delivery system, secondary toxicity effects of the chemotherapy, such as liver or kidney damage and pulmonary toxicity are avoided. The present invention provides for drug delivery systems with a continuous, steady delivery of chemotherapy drugs, which provides longer release than any comparative commercial product, such as Gliadel® (release of chemo agent for 1 week after implantation).

The present invention also incorporates the discovery that changing the fiber morphology (porous fibers, branched/networked/crosslinked fibers, beaded fibers, etc.) changed the rate of release, but not necessarily the rate of degradation. From this, the release rate can be manipulates to obtain a predetermined release profile (i.e., sustained, burst, S curve, exponentially increasing or exponentially decreasing).

The electrospun fiber webs of the present invention are also unexpectedly strong despite their high porosity and thus are suitable for the repair of weakened blood vessel walls such as are found in aneurisms. Therefore, another aspect of the invention provides a method for repairing in a patient a blood vessel characterized by a weakened wall by applying to the interior of the weakened blood vessel wall an electro-spun biodegradable resorbable polymeric fiber matrix having an interfibrillar space of at least 65% by volume.

### DESCRIPTION OF THE FIGURES

FIG. 1 (a) depicts bulk erosion drug delivery. FIG. 1 (b) depicts surface degradation for drug release. L. Brannon-Peppas, Medical Plastics and Biomaterials Magazine, p. 34, (1997);

FIG. 2 depicts an Electrospinning device;

FIG. 3 depicts the fiber behavior in an electrospinning process by (a) Fluid jet, (b) Spraying, (c) Whipping motion. Shin et al., Appl. Phys. Let., 78(8), February 2001;

FIG. 4 provides a comparison of supernatant pH changes during 3 months incubation of the electrospun 85/15 μm fiber discs, 75/25 μm fiber discs, 50/50 μm fiber discs; and 2 month incubation of 50/50 nm fiber discs, and 50/50 beaded morphology discs (n=5);

FIG. 5 provides the loss in disc diameter during incubation of electrospun 85/15 μm fiber discs, 75/25 μm fiber discs, 50/50 μm fiber discs, 50/50 nm fiber discs, and 50/50 discs composed of both fiber and beaded morphology (n=7);

FIG. 6 provides the molecular weight loss during incubation of electrospun 85/15 μm fiber discs, 75/25 μm fiber discs, 50/50 μm fiber discs, 50/50 nm fiber discs, and 50/50 discs composed of both fiber and beaded morphology (n=1);

FIG. 7 provides the tracking of water uptake during the degradation process. Electrospun mats types tracked were 85/15 μm fiber discs, 75/25 μm fiber discs, 50/50 μm fiber discs, 50/50 nm fiber discs, and 50/50 discs of both fiber and beaded morphology (n=1);

FIG. 8 provides for the tracking of changes in the void space of incubated electrospun mats. Intrafibrillar spaces were ascertained using the formula published by Barnes et al., (2006), J. Engineer. Fibers and Fabrics, 1(2), 16-29;

FIG. 9 provides for tracking surface area to volume ratio of incubated electrospun mats. Ratio was calculated using formula published by Barnes et al., Id.;

FIG. 10 depicts the surface area to volume ratio for electrospun 50/50 DLPLGA nano-fiber discs. Ratio was calculated using formula published by Barnes et al., Id.;

FIG. 11 depicts the change in T_{g} as a function of aging time for 50/50 DLPLGA electrospun polymer discs (micron, nano, and beaded fibers) incubated in stagnant PBS at 37° C.;

FIG. 12 depicts enthalpic relaxation as a function of aging time for 50/50 DLPLGA polymer electrospun discs (micron, nano, and beaded fibers) incubated in stagnant PBS at 37° C.;

FIG. 13 depicts a comparison of supernatant pH changes between test tubes incubated with one disc versus two discs, of equal weight for electrospun 85/15, 75/25, and 50/50 μm fiber discs;

FIG. 14 depicts a comparison of water uptake in test tubes incubated with single versus double disc samples, of equal weight for electrospun 85/15, 75/25, and 50/50 μm fiber discs;

FIG. 15 depicts the changes in supernatant pH for drug loaded electrospun (e-spun) discs. Samples tested were 1 wt % Guaifenesin in stagnant solution (1 wt % G Set1) and dynamic solution (1 wt % G Set2); 3 wt % Guaifenesin in stagnant solution (3 wt % G Set1) and dynamic solution (3 wt % G Set2); and 1 wt % Beta-Estradiol in stagnant solution (1 wt % E Set1) and dynamic solution (1 wt % E Set2) (n=4);

FIG. 16 depicts water uptake of drug loaded discs. Samples tested were 1 wt % Guaifenesin in stagnant solution (1 wt % G Set1) and dynamic solution (1 wt % G Set2); 3 wt % Guaifenesin in stagnant solution (3 wt % G Set1) and dynamic solution (3 wt % G Set2); and 1 wt % Beta-Estradiol in stagnant solution (1 wt % E Set1) and dynamic solution (1 wt % E Set2) (n=1);

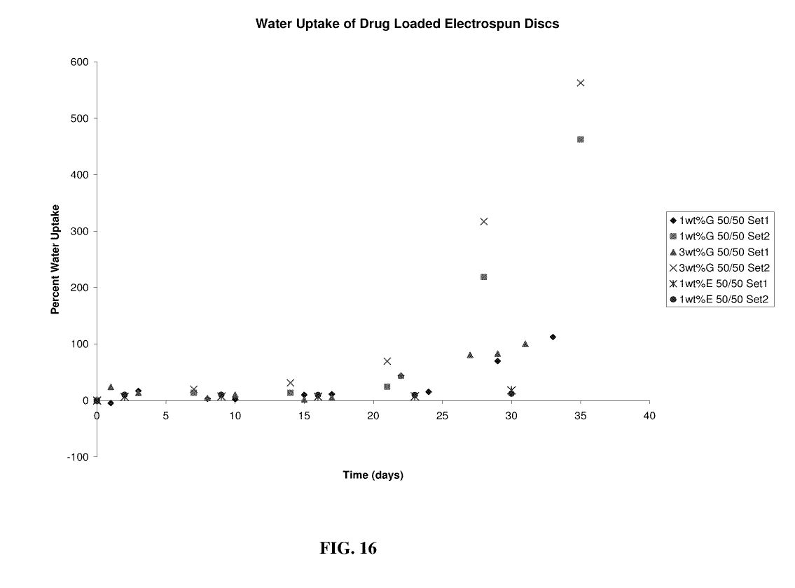

FIG. 17 depicts the loss in diameter size of drug loaded disc samples starting at 12.7 mm. Samples tested were 1 wt % Guaifenesin in stagnant solution (1 wt % G Set 1) and dynamic solution (1 wt % G Set2); 3 wt % Guaifenesin in stagnant solution (3 wt % G Set1) and dynamic solution (3 wt % G Set2); and 1 wt % Beta-Estradiol in stagnant solution (1 wt % E Set1) and dynamic solution (1 wt % E Set2) (n=4);

FIG. 18 depicts molecular weight changes as a function of time for drug loaded samples incubated. Samples tested were 1 wt % Guaifenesin in stagnant solution (1G Set1) and dynamic solution (1G Set2); 3 wt % Guaifenesin in stagnant solution (3G Set1) and dynamic solution (3G Set2); and 1 wt % Beta-Estradiol in stagnant solution (1E Set1) and dynamic solution (1E Set2) (n=1);

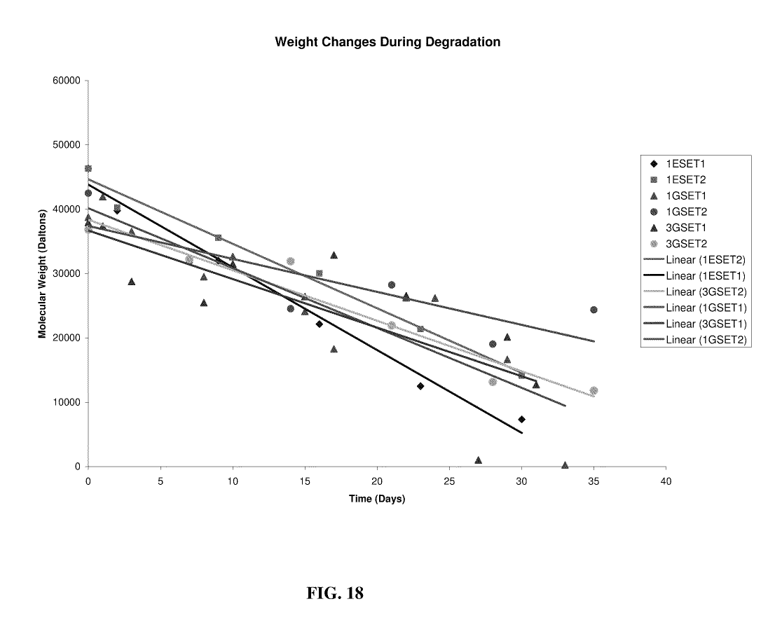

FIG. 19 depicts interfibrillar space changes of incubated drug loaded discs as a function of time, calculated using formula by Barnes et al. Id. Samples tested were 1 wt % Guaifenesin in stagnant solution (1GS1) and dynamic solution (1GS2); 3 wt % Guaifenesin in stagnant solution (3GS1) and dynamic solution (3GS2); and 1 wt % Beta-Estradiol in stagnant solution (1ES1) and dynamic solution (1ES2);

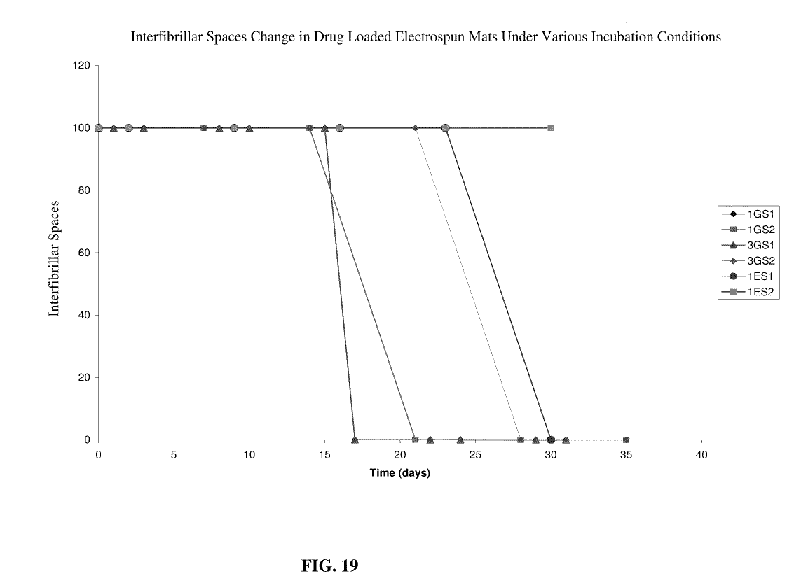

FIG. 20 depicts the surface area to volume ratio of incubated drug loaded electrospun discs calculated using formula by Barnes et al. supra. Samples tested were 1 wt % Guaifenesin in stagnant solution (1GS1) and dynamic solution (1GS2); 3 wt % Guaifenesin in stagnant solution (3GS1) and dynamic solution (3GS2); and 1 wt % Beta-Estradiol in stagnant solution (1ES1) and dynamic solution (1ES2);

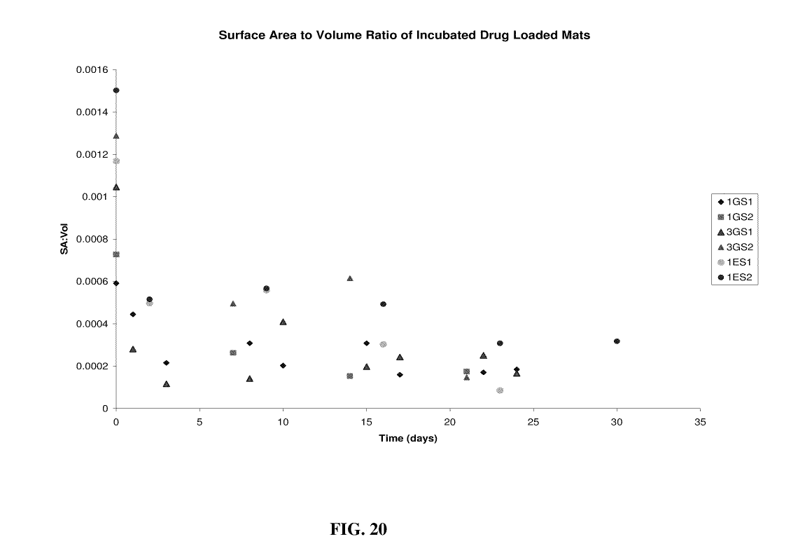

FIG. 21 depicts the detection of T_{g} as a function of aging time for electrospun polymer-drug discs incubated in PBS at 37° C. Samples tested were 1 wt % Guaifenesin in stagnant solution (1GS1) and dynamic solution (1GS2); 3 wt % Guaifenesin in stagnant solution (3GS1) and dynamic solution (3GS2); and 1 wt % Beta-Estradiol in stagnant solution (1ES1) and dynamic solution (1ES2);

FIG. 22 depicts the enthalpic relaxation measurements as a function of aging time for electrospun polymer-drug discs incubated in PBS at 37° C. Samples tested were 1 wt % Guaifenesin in stagnant solution (1GS1) and dynamic solution (1GS2); 3 wt % Guaifenesin in stagnant solution (3GS1) and dynamic solution (3GS2); and 1 wt % Beta-Estradiol in stagnant solution (1ES1) and dynamic solution (1ES2);

FIG. 23 depicts the degree of molecular weight changes as a function of time for incubated drug loaded electrospun discs after 7 month storage (n=1). Samples tested were 1 wt % Guaifenesin in stagnant solution (Mini 1GS1) and dynamic solution (Mini 1GS2); 3 wt % Guaifenesin in stagnant solution (Mini 3GS1) and dynamic solution (Mini 3GS2); and 1 wt % Beta-Estradiol in stagnant solution (*1ES1 in BP) and dynamic solution (*1ES2 in BP). All Guaifenesin samples incubated in PBS, Beta-Estradiol samples incubated in blood plasma (BP);

FIG. 24 depicts changes in supernatant pH as a function of time for incubated drug loaded discs after storage for 7 months. Only the Guaifenesin samples incubated in PBS were tested. The pH meter could not mark a clear reading on the pH value for the incubated Beta-Estradiol samples. Samples tested were 1 wt % Guaifenesin in stagnant (Mini 1GS1) and dynamic solution (Mini 1GS2); 3 wt % Guaifenesin in stagnant (Mini 3GS1) and dynamic soln (Mini 3GS2) (n=2);

FIG. 25 depicts the diameter loss as a function of time for incubated drug loaded electrospun discs stored for 7 months, with an initial diameter of 12.7 mm. Samples tested were 1 wt % Guaifenesin in stagnant solution (Mini 1GS1) and dynamic solution (Mini 1GS2); 3 wt % Guaifenesin in stagnant solution (Mini 3GS1) and dynamic solution (Mini 3GS2); 1 wt % Beta-Estradiol in stagnant (*1ES1 in BP) and dynamic (*1ES2 in BP) solution of blood plasma (BP) (n=2);

FIG. 26 depicts the water uptake of incubated drug loaded electrospun discs stored for 7 months. Samples tested were 1 wt % Guaifenesin in stagnant solution (Mini 1GS1) and dynamic solution (Mini 1GS2); 3 wt % Guaifenesin in stagnant solution (Mini 3GS1) and dynamic solution (Mini 3GS2); 1 wt % Beta-Estradiol in stagnant (*Mini 1ES1 in BP) and dynamic (*Mini 1ES2 in BP) solution of blood plasma (BP) (n=1);

FIG. 27 depicts the degree of interfibrillar spaces change of incubated drug loaded electrospun discs stored for 7 months. Samples tested were 1 wt % Guaifenesin in stagnant solution (Mini 1GS1) and dynamic solution (Mini 1GS2); 3 wt % Guaifenesin in stagnant solution (Mini 3GS1) and dynamic solution (Mini 3GS2); 1 wt % Beta-Estradiol in stagnant (Mini 1ES1 in BP*) and dynamic (Mini 1ES2 in BP*) solution of blood plasma (BP);

FIG. 28 depicts the surface area to volume ratio of incubated drug loaded electrospun discs stored for 7 months. Samples tested were 1 wt % Guaifenesin in stagnant solution (Mini 1GS1) and dynamic solution (Mini 1GS2); 3 wt % Guaifenesin in stagnant solution (Mini 3GS1) and dynamic solution (Mini 3GS2); 1 wt % Beta-Estradiol in stagnant (Mini 1ES1 in BP*) and dynamic (Mini 1ES2 in BP*) solution of blood plasma (BP);

FIG. 29 depicts a comparison of water uptake for mini study of 5 wt % Guaifenesin loaded discs and 1 wt % Guaifenesin/1 wt % Beta-Estradiol loaded discs (n=1). 1 wt % and 3 wt % Guaifenesin data points and 1 wt % Beta-Estradiol from the original drug study are included for comparison;

FIG. 30 depicts the molecular weight changes for mini study of 5 wt % Guaifenesin loaded discs and 1 wt % Guaifenesin/1 wt % Beta-Estradiol loaded discs (n=1). 1 wt % and 3 wt % Guaifenesin data points and 1 wt % Beta-Estradiol from the original drug study are included for comparison;

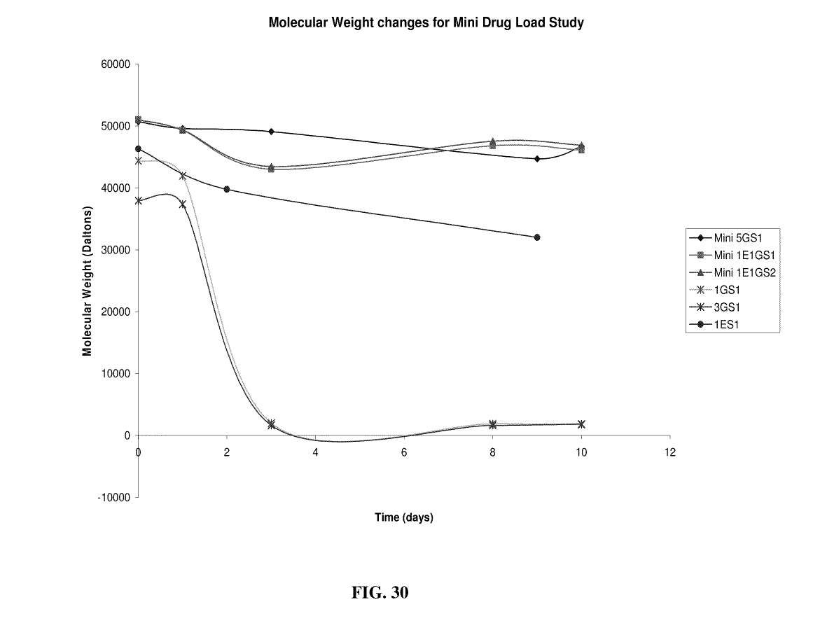

FIG. 31 depicts the changes in supernatant pH mini study of 5 wt % Guaifenesin loaded discs and 1 wt % Guaifenesin/1 wt % Beta-Estradiol loaded discs (n=2). 1 wt % and 3 wt % Guaifenesin data points and 1 wt % Beta-Estradiol from the original drug study are included for comparison;

FIG. 32 depicts the degree of loss in diameter from 12.7 mm mini study of 5 wt % Guaifenesin loaded discs and 1 wt % Guaifenesin/1 wt % Beta-Estradiol loaded discs (n=1). 1 wt % and 3 wt % Guaifenesin data points and 1 wt % Beta-Estradiol from the original drug study are included for comparison;

FIG. 33 depicts the interfibrillar spaces change as a function of time mini study of 5 wt % Guaifenesin loaded discs (Mini 5GS1) and 1 wt % Guaifenesin/1 wt % Beta-Estradiol loaded discs (Mini 1E1GS1, Mini 1E1GS2);

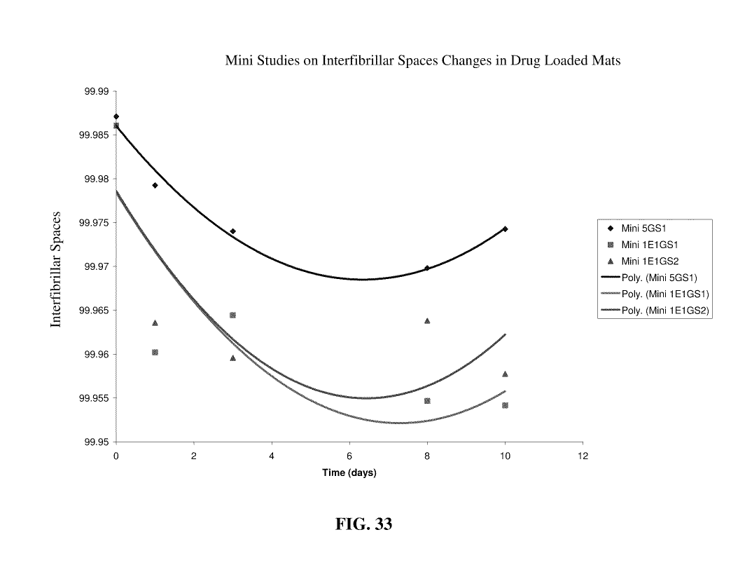

FIG. 34 depicts the changes of surface area to volume ratio per time for mini study of 5 wt % Guaifenesin loaded discs (Mini 5GS1) and 1 wt % Guaifenesin/1 wt % Beta-Estradiol loaded discs (Mini 1E1 GS1, Mini 1E1GS2);

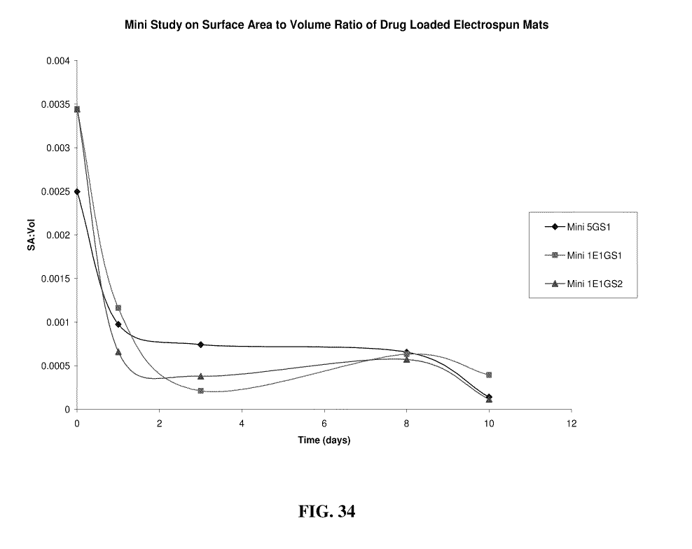

FIG. 35 depicts the release profile of all electrospun drug delivery systems incubated in stagnant solution. Samples analyzed were 1 wt % Guaifenesin (1GS1); 3 wt % Guaifenesin (3GS1); 1 wt % Beta-Estradiol (1ES1); 5 wt % Guaifenesin (Mini 5GS1); 1 wt % Guaifenesin/1 wt % Beta-Estradiol (Mini 1E1 GS1) (n=4);

FIG. 36 depicts the release profile of all electrospun drug delivery systems incubated in dynamic solution. Samples analyzed were 1 wt % Guaifenesin (1GS2); 3 wt % Guaifenesin (3GS2); 1 wt % Beta-Estradiol (1ES2); 1 wt % Guaifenesin/1 wt % Beta-Estradiol (Mini 1E1 GS2) (n=4);

FIG. 37 depicts the enthalpic relaxation as a function of aging time with respect to drug release of electrospun drug loaded discs. Samples compared were 1 wt % Beta-Estradiol (1ES2) and 1 wt % Guaifenesin (1GS2) in dynamic PBS at 37° C. Enthalpic relaxation is the catalyst for drug release;

FIG. 38 depicts the release profile of all electrospun drug delivery systems stored for 7 months then incubated in stagnant solution. Samples analyzed were 1 wt % Guaifenesin (1GS1); 3 wt % Guaifenesin (3GS1); 1 wt % Beta-Estradiol (1ES1 in BP*) (n=2). The 1 wt % Beta-Estradiol was incubated in blood plasma instead of PBS;

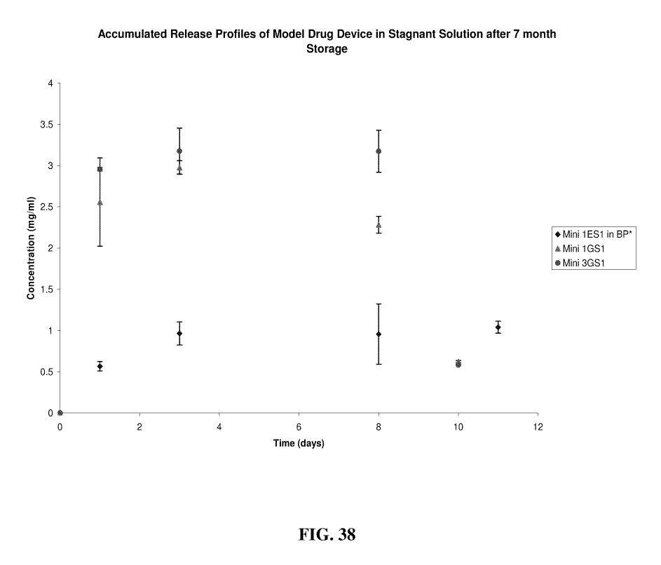

FIG. 39 depicts the release profile of all electrospun drug delivery systems stored for 7 months then incubated in dynamic solution. Samples analyzed were 1 wt % Guaifenesin (1GS2); 3 wt % Guaifenesin (3GS2); 1 wt % Beta-Estradiol (1ES2 in BP*) (n=2). The 1 wt % Beta-Estradiol was incubated in blood plasma instead of PBS;

FIG. 40 is a representative total heat flow DSC heating thermograms for high and low Mw PLGA microspheres in the glass transition region (labels next to curves). Dotted lines extrapolate a baseline to delineate the overheating peak area, integrated to give the enthalpy relaxation. Dashed lines extrapolate the baseline and steepest point on the curve to measure the onset T_{g}. Rouse et al., Intern. J. Pharm., 339, 112-120, (2007);

FIG. 41 is the linear regression of the logarithm of the physical ageing time for the PLGA microsphere formulations and the enthalpy relaxation at 35° C. (r²-values are 0.99 for each curve). Emulsifiers used in the primary emulsion were PVA (up triangles), BSA (down triangles) and Triton X-100 (circles). Rouse et al., Intern. J. Pharm., 339, 112-120, (2007);

FIG. 42 is a depiction of in vitro release profiles of estradiol loaded PLGA (50:50) nanoparticles of different molecular weights with DMAB as stabilizer in pH 7.4 phosphate buffer. Data points shown are mean±standard deviation (n=3). Mittal et al., J. Cont. Rel., 119, 77-85, 2007;

FIG. 43 is a depiction of In vitro release profiles of estradiol loaded PLGA nanoparticles of different copolymer compositions with DMAB as stabilizer in pH 7.4 phosphate buffer. Each data point is a mean of three values. Mittal et al., J. Cont. Rel., 119, 77-85, 2007

FIG. 44 is the DSC thermograms of BCNU, PLGA and BCNU-loaded PLGA microparticles: (a) BCNU; (b) PLGA 20k; (c) PLGA 90k; (d) PLGA 20k/BCNU 10%; and (e) PLGA 90k/BCNU 10%. Seong et al., Intern. J. Pharma., 251, 1-12, (2003); and

FIG. 45 depicts the effect of BCNU loading amount on BCNU release from PLGA wafers: (a) PLGA 20k and (b) PLGA 90k. Seong et al., Intern. J. Pharma., 251, 1-12, (2003).

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs, specially in the field of synthetic chemistry, pharmacology and cosmetology. The terminology used in the description of the invention herein is for describing particular embodiments only and is not intended to be limiting of the invention. All publications, patent applications, patents, figures and other references mentioned herein are expressly incorporated by reference in their entirety.

The term “matrix” denotes the physical structure of the polymeric moiety. Solid matrices essentially retain the active agent in a manner preventing release of the agent until the polymer erodes, decomposes or broken down to its precursor material.

The terms “vehicle” and “carrier” denote an ingredient that is included in a composition such as a pharmaceutical or cosmetic preparation for reasons other than a therapeutic or other biological effect. Functions served by vehicles and carriers include transporting an active agent to a site of interest, controlling the rate of access to, or release of, the active agent by sequestration, surface wetting, buffering or other means, and facilitating the application of the agent to the region where its activity is needed. Such agents include physiologic salts, surfactants, chelators, etc.

The terms “controlled release”, “sustained release” and similar terms are used to denote a mode of active agent delivery that occurs when the active agent is released from the polymeric matrix at an ascertainable and manipulatable rate over a period of time, rather than dispersed immediately upon application. Controlled or sustained release may extend for hours, days or months, and may vary as a function of numerous factors. In the present invention, an important determinant of the rate of delivery is the rate of hydrolysis of the linkages between and within the units of the polymer. The rate of hydrolysis in turn may be controlled by the composition of the polymeric matrix and the number of hydrolyzable bonds in the polymer. Other factors include implant size, length of the electrospun fibers, acidity of the medium (either internal or external to the matrix), solubility of the active agent in the matrix, molecular weight and charge density of the active agent.

The term “therapeutic active agent” is intended to include any compound or mixture of compounds which produces a beneficial or useful for medical treatment. They include contrast agents and radio-opaque materials as well as active agents. The recitation of “therapeutic active agent” ais distinguishable from such components as vehicles, polymeric carriers, diluents, lubricants, minerals, and other formulating aids. Examples of “therapeutic active agents” are antibodies, anti-infectives (including antibiotics, antivirals, fungicides, scabicides or pediculicides), vaccines, hormones (for example, estrogens, progestins, androgens, adrenocortical steroids, insulin, erythropoietin and the like), vitamins, enzymes, proteins, naturally occurring or bioengineered substances, anti-inflammatory agents (for example, aspirin, ibuprofen, naproxen and the like), fertility control agents, chemotherapeutic and anti-neoplastic agents (for example, mechlorethamine, cyclophosphamide, 5-fluorouracil, thioguanine, carmustine, lomustine, melphalan, chlorambucil, streptozocin, methotrexate, vincristine, bleomycin, vinblastine, vindesine, dactinomycin, daunorubicin, doxorubicin, tamoxifen and the like), anti-proliferative agents (e.g. rapamycin, paclitaxel, or plasminogen activator inhibitors or the like), and anti-epilepsy agents (e.g. phenyloin, ethotoin and the like).

A “therapeutic implant” or “implant” as used herein is a device used for placement in a tissue defect in a patient (human or animal) to provide further treating or healing of the defective area.

The terms “biodegradable,” and “bioerodible” refer to the degradation or disassembly of a polymer by action of a biological environment by the way of linkage breakdown by such mechanisms including hydrolysis, enzyme, pH or temperature degradation. The term “polymer hydrolysis”, for the purpose of this invention, refers to the hydrolysis of the linkages between and within the units of the polymeric moiety.

Polymers known in the art for producing biodegradable implant materials include, but are not limited to polyglycolide (PGA), copolymers of glycolide such as glycolide/L-lactide copolymers (PGA/PLLA), glycolide/trimethylene carbonate copolymers (PGA/TMC); poly-lactides (PLA), stereocopolymers of PLA such as poly-L-lactide (PLLA), Poly-DL-lactide (PDLLA), L-lactide/DL-lactide copolymers; copolymers of PLA such as lactide/tetramethyl-glycolide copolymers, and lactide/trimethylene carbonate copolymers, preferably the FDA approved biodegradable synthetic polymer which have had a history of success in medical device research.

The biodegradable polymeric implant polymeric fiber matrix can be prepared in variety of shapes or form including cylinders, wafers, spheres, strips, films, and irregularly-shaped implants. A therapeutic implant is a device used for placement in a tissue defect in a patient (human or animal) to encourage ingrowth of tissue and healing of the defect, or for delivery of a therapeutic agent to the interior walls of the defect.

Preferred biodegradable polymers for use in making the materials of this invention are known to the art, including aliphatic polyesters, preferably polymers of polylactic acid (PLA), polyglycolic acid (PGA) and mixtures and copolymers thereof, more preferably about 50:50 to about 85:15 molar ratio copolymers of D,L-PLA and PGA, and most preferably D,L-PLA and PGA copolymers in a molar ratio of D,L-PLA to PGA between about 55:45 and about 75:25. Single enantiomers of PLA may also be used, preferably L-PLA, either alone or in combination with PGA.

Preferably the polymeric implant material has a molecular weight between about 500 and about 1,000,000 Daltons, more preferably between about 5,000 and about 400,000 Daltons, and most preferably between about 55,000 and about 250,000 Daltons. The molecular weight is measured by conventional techniques for the given polymer,

As used herein “local or “localized” delivery is non-systemic delivery wherein one or more therapeutic agents are deposited within a tissue, for example, a nerve root of the nervous system or a region of the brain, or in close proximity (within about 10 cm, or preferably within about 5 cm, of the site of interest). The delivery is done by direct and local administration of the delivery system to allow release of the therapeutic active agents

As used herein “interfibrillar spaces” or “IFS” is meant the spaces between fibers in the electrospun delivery system, disc constructs, implant or nanofiber matrix.

Electrospinning is the process of charging a drop of polymer in solution with high (5-30 kV) voltage causing the deformation of a Taylor cone into polymer fibers with nanoscale diameter. The term “electrospinning” was derived from “electrostatic spinning” a term first used by Formhals in 1934 in a United States patent. Shin, Y. M. et al. (2001), Appl. Phys. Lett., 78:8.

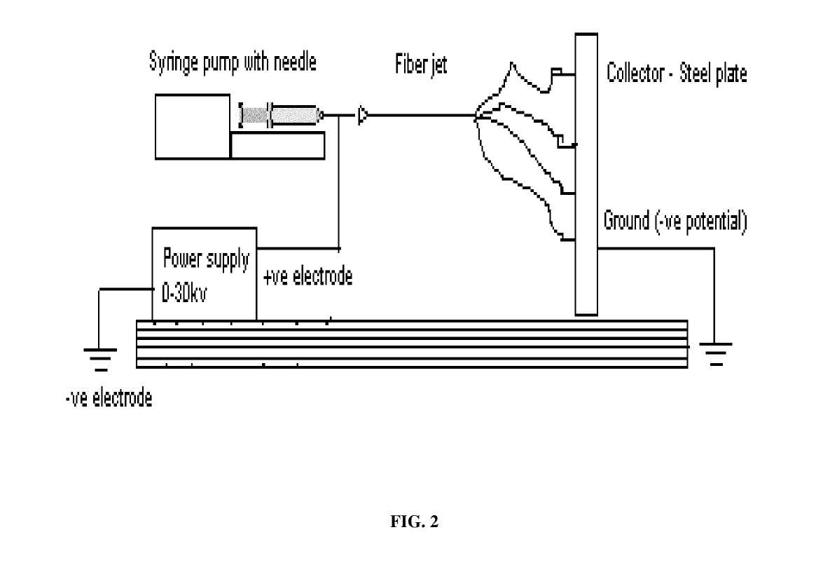

The process was adapted in the present invention to collect polymer nanofibers as a nonwoven sheet on a grounded plate below the capillary [FIG. 2]. In general, increasing the grounded surface distance, decreases fiber diameter; increasing electric potential (kV), decreases fiber diameter to a critical point; increasing flow rate, increases fiber diameter; increasing wt % concentration, increases fiber diameter; and decreasing the conduit size decreases fiber diameter. Physiological salts or buffers can be used to modify the electric potential of the solution and other electrospinning properties.

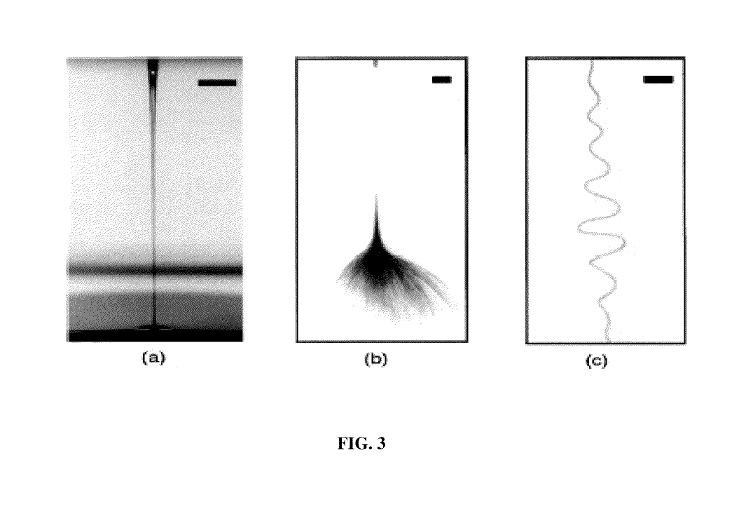

The process of applying an electric fields to a polymer solution (electrostatic charging of the fluid at the tip of the conduit) results in the formation of a Taylor cone, in which multiple filaments are ejected to produce nonwoven materials that have a interfibrillar spaces factor, high surface area, and fineness and uniformity. The filaments are the result of the jet accelerating and thinning in the electric field, a process first described as “splaying.” Further studies suggested that the most important element operative during electrospinning is the rapid growth of a non-axisymmetric, or “whipping,” instability that causes bending and stretching of the jet (FIG. 3 (a)). Shin, Y. M. et al. (2001), Appl. Phys. Lett., 78:8.

The unstable region of the jet (FIG. 3(b)) viewed at exposure times down to the millisecond, has the appearance of an “inverted cone,” while in FIG. 3 (c), using high-speed photography, a single, rapidly whipping jet was observed by Shin et al. supra. The whipping frequency is at such levels that the jet appears to be splitting into multiple filaments, hence the initial term “splaying.” Shin et al, supra.

Hohman et al. demonstrated three different modes which are unstable: (1) the Rayleigh mode, which is the axisymmetric extension of the classical Rayleigh instability when electrical effects are important; (2) the axisymmetric conducting mode; and (3) the whipping conducting mode. Source: Hohman, et al. (2001). Physics of Fluids, 13:8.

The latter are dubbed “conducting modes” because they only exist when the conductivity of the fluid is finite. Source Hohman, M. M., et al supra. The classical Rayleigh instability occurs due to surface tension, which acts to break a cylindrical jet into a stream of droplets having the same volume, but lower surface area. Hohman et al. demonstrated that the dominant instability strongly depends on the fluid parameters of the fluid jet (viscosity, dielectric constant, and conductivity) and also the static charge density on the jet. Id.

The present invention provides methods, systems and reagents for facilitating implantation of a drug delivery system into a subject by contacting tissues to be treated with a polymeric electrospun fiber matrix according to the present invention loaded with a therapeutic active agent. The drug delivery systems are preferably local implants designed for placement and local administration into a tissue defect within soft or hard tissues such as neural tissues of the brain and spinal cord, synovial joint tissue, the spinal disc space, the spinal canal or the surrounding soft tissue.

A drug delivery system in the form of an implant of the present invention comprises a physical structure inherently capable of implantation and retention in a desired location of a subject, such as for example, a brain lesion or post surgical brain scar. In another embodiment the biodegradable and/or bioabsorbable electrospun fiber contains a material derived from biological tissue, e.g., collagen, gelatin, polypeptides, proteins, hyaluronic acid and derivatives or synthetic biopolymers. The fibers of different biodegradable and/or bioabsorbable materials can include fibers having different chemical composition, such as different polymeric materials, different molecular weights of the same polymeric material, different blends of polymers, materials having different additives or materials having different concentration of additives.

In at least one embodiment of the present invention, the delivery system can be in the form of an implant or a disc mat formed from electrospun nanoparticles or nano-fibers having different diameters in the range from one nanometer up to about four microns, preferably between about five nm and about 3.5 microns, more preferably between about ten nm and about 2 microns, even more preferably between about 50 and about 1000 nanometers and most preferably between about 100 and about 750 nanometers. The delivery system of the present invention can contain particles or fibers of having diameters less than five nanometers combined with particle's or fibers having diameters greater than 750 nanometers. In another aspect of this embodiment, the delivery system can be a controlled release implant.

In another embodiment of the present invention, a biodegradable controlled release implant of electrospun biodegradable polymeric fibers is provided that contains at least one therapeutic agent selected from anti-infective agents, anti-histamines, anti-tussive agents, chemotherapeutic agents, contraceptives, corticosteroids and expectorants. In a more preferred embodiment, the implant contains nanosized particles or fibers having a diameter in the range from about one nanometer up to about two microns, preferably between about 5 nm and about 3.5 microns, more preferably between about 10 nm and about 2 microns, even more preferably between about 50 and about 1000 nanometers and most preferably between about 100 and about 750 nanometers. The delivery system of present invention can contain particles or fibers having diameters less than 5 nanometers combined with particles or fibers having diameters greater than 750 nanometers.

In an embodiment, the polymeric product has an IFS that enhances adhesion of the product to tissue surfaces. In another embodiment, the delivery systems may be stored for periods of at least four months, preferably at least 7 months, and even greater, without any change to the release profile or extent of drug release. Accordingly, in at least one aspect of the present invention a biodegradable drug delivery implant is provided characterized by an electro-spun biodegradable polymeric fiber matrix in nanosize scale with at least one therapeutic active agent incorporated into the polymer fibers. In a more preferred aspect of this invention, the therapeutic active agent is an anti-infective agent, an anti-histamine, an anti-tussive agent, a chemotherapeutic agent, a contraceptive, a corticosteroid or an expectorant. In another embodiment, the electrospun polymer fiber drug delivery system is stored under reduced atmospheric pressure wherein the pressure ranges from 350 to 760 mm Hg.

In yet another aspect of the present invention, the delivery system of the present invention, shows a interfibrillar spaces change of not more than 5%, 4%, 3%, 2%, 1%, or 0.5% of its original volume after at least four months, and preferably after at least seven months storage. In another embodiment, the IFS is at least 65% of the total volume of the construct or the delivery system, preferably at least 75% of the total volume, more preferably in the range of 85% to 99.9% of the total volume, and most preferably in the range of 96 to 99.5 percent of the total volume. In another aspect of the present invention, storage at temperature of 25° C. to 37° C. of the product for up to at least months, and preferably up to at least seven months, does not alter the polymer crystallinity, interfibrillar space volume or surface area to volume ratio of the electropsun fiber system.

In another embodiment the present invention provides accelerated polymeric degradation of a nano-size polymer fiber matrix by incorporating into a matrix one or more of: hydrophilic moieties in the polymer backbone or end groups, greater reactivity among hydrolytic groups in the backbone, less polymer crystallinity, greater polymer fiber interfibrillar spaces, irregular polymer fiber morphology in the matrix and smaller finished device size. In yet another embodiment, the physiological salts or buffers that are added to polymer solutions to adjust the solution potential also improve the wetability or adhesiveness of the final polymeric matrix with fluids such as blood plasma, etc.

In another embodiment, the polymeric matrix of the present delivery system is a polyester of poly(glycolic acid), poly(lactic acid), poly(alpha or beta hydroxyacid) esters and copolymers and mixtures thereof. In a more preferred embodiment, the polymer is a poly (lactic acid), poly(glycolic acid) or a copolymer thereof, and in a most preferred embodiment, the polymer ratio of lactic acid to glycolic acid ranges between about 85:15 and about 50:50.

In one embodiment of the present invention, the therapeutic agent can be hydrophilic or hydrophobic. In an embodiment the therapeutic agent is a chemotherapeutic agent. In a more specific embodiment, the chemotheraepeutic agent is an alkylating agent, a nitrosourea, an alkylsulfonates, an antimetabolite, a pyrimidine analogue, a purine analogue, an antimimotic agent, an antibiotic, a platinum coordination complex, an aromatase inhibitor, a gonadotropin analog, or biological modifier, or combinations thereof. In an even more specific embodiment, the therapeutic agent is BCNU, doxorubicin, mitomycin, 5-FU, methotrexate, busulfan, cisplatin, hydroxyurea, procarbozine, paclitaxel, doce-taxel, vincristine, or mercaptopurine.

Accordingly, methods are provided for using the delivery system of the present invention for treatment of post surgical lesions containing residual tumor cells. The present invention is also directed to methods of treating patients at risk of relapse from a primary solid tumor. At least another aspect of the present invention is directed to methods of treating residual tumor cells present in a post-solid tumor resection surgery wherein the solid tumor or the cancerous tissue is under a dynamic physiologic conditions and/or exposed to continuous flow of the cerebrospinal fluid (CSF). In this aspect of the present invention, the inventors disclose a method of controlled drug delivery to ensure that the entire excised or resected area is exposed to adequate anti-tumor drug regimen. In yet another aspect of the present invention, the presently disclosed methodology will minimize the risk of relapse and/or reoccurrence of cancerous tissue due to residual tumor cells infiltration into other regions of the body.

In another aspect, the present invention is directed to delivery systems that can be used to ablate diseased tissue (e.g., tumors, etc.) by cutting off its blood supply. In a preferred embodiment, the present invention is directed to methods of treating brain tumor and malignancies caused by such conditions including but not limited to glioblastoma multiforme. In a more preferred embodiment, the delivery system is applied to regions of interest after excision of the pathologic tissue or tumor.

The present invention is also directed to methods of preparing sustained and/or controlled release drug delivery systems. In one embodiment, the process of making such a system provides for subjecting a mixture of a biodegradable polymer, an organic solvent and a therapeutic agent to a flash-flow melt-spinning mechanism, thereby embedding a therapeutic active agent into the polymeric mixture under conditions suitable for producing an unwoven polymeric fiber matrix capable of releasing the active in an animal for a desired period of time. In a more specific embodiment, the present invention employs a polyester such as poly(glycolic acid), poly(lactic acid), poly(alpha- or beta-hydroxyacid) esters and copolymers and mixtures approved by the FDA as a suitable biodegradable fiber matrix polymers. The present invention includes embodiments in which the polymeric fiber matrix is part of a drug reservoir for a transdermal drug delivery device.

In yet another embodiment of the present invention, the delivery system is a tissue scaffold for wound closure or tissue repair fabricated from various types of nanoparticles, nanofibers and nanosize disc mats, having different diameters in the range from about one nanometer up to about one micron, preferably between about 10 nm and about 950 nanometers, more preferably between about 25 and about 750 nanometers and most preferably between about 50 and about 500 nanometers. The tissue scaffold of present invention can contain particles or nanofibers of having diameters less than 50 nanometers combined with particles or fibers having diameters greater than 500 nanometers. The scaffold may optionally contain therapeutic agents to promote wound healing, infection prevention or tissue regeneration.

Electrospun polymer fiber matrices according to the resent invention are unexpectedly strong and can be used for tissue repair. Therapeutic agents are optionally included in polymer fiber matrices for tissue repair according to the present invention.

In one embodiment, the fiber matrix is dimensioned to repair weakened blood vessel walls such as may be found in an aneurism such as a cranial aneurism and may optionally contain a thrombogenic agent to stop fluid leakage from the blood vessel wall. In another aspect of the present invention; a method is provided for treating an aneurysm sac and/or encourage clot formation so that blood flow into the aneurysm ceases. Accordingly, at least another aspect of the present invention is directed to methods of treating vascular abnormalities such as AVM's and AVF's by forming a plug or clot to control/reroute blood flow to permit proper tissue perfusion.

In one aspect of the present invention two models of active agents were used in certain exemplary embodiments. The active agents exemplified herein include Guaifenesin and Beta-Estradiol. These were chosen for illustrative purposes only and active agents for use with the present invention are not in any way limited to these two agents. BCNU is a proper model for this process whereas the two selected model drugs of choice were simulants.

BCNU (bis-chloronitrosourea, carmustine) is a nitrosourea that has the ability to cross the blood-brain barrier due to its chemical properties as well as its size. It has a molecular weight (MW) of 214 g/mole and its exact mechanism in chemotherapy is unknown. It is excreted by the body through urine and CO₂. With intravenous infusion of BCNU doses ranging from 30 to 170 mg/m², the average terminal half-life, clearance, and steady-state volume of distribution are 22 minutes, 56 mL/min/kg, and 3.25 L/kg, respectively. It has a melting temperature (Tₘ) of 31° C. and an experimental water solubility less than 0.1 g/100 mL. Based on clinical data, evidence of BCNU release via the Gliadel® wafer is detectable up to 1 week. Source: Westphal et al., supra. Neuro Oncology, 79-88 (2003).

More than 70% of the Gliadel® wafer degrades by three weeks and trace elements of the wafer are detected up to twelve weeks in the patient surgical cavity. Westphal et al., supra. See also Katz, (January 2001), Medical Device and Diagnostic Industry Magazine, 122.

Guaifenesin ((2RS)-3-(2-Methoxyphenoxy)propane-1,2-diol or glyceryl guaiacolate, is an expectorant drug to assist the bringing up of phlegm from the airways in acute respiratory tract infections. It is used in the treatment of colds, asthma, gout, fibromyalgia, and relaxation of muscles. Its chemical formula is C₁₀H₁₄O₄, and it has a MW of 198 g/mole and a melting point of 78.5° C. Dosages range between 300 mg-2.4 g per day depending on the age and condition of the patient. The half-life is approximately 1 hour, metabolized by oxidation to the urinary metabolite β-2-(methoxyphenoxy)lactic acid which is excreted through urine. It has a water solubility of 50 mg/mL and toxic overdose has occurred.

Beta-Estradiol (E2 or 17β-estradiol) is a commonly used female hormone for hormone replacement therapy. Its chemical formula is C₁₈H₂₄O₂, and it has a MW of 272 g/mole and a melting temperature range of 175-177° C. As a medication, it is packaged in various forms (oral, gels, injections) and comes in various dosage forms but generally 1 mg per day is sufficient for a variety of uses. It is 97-99% protein bound, metabolized by the liver and excreted in urine. It has a water solubility of 3.6 mg/L and a half life of 36 hours.

At least one aspect of the present invention includes small particles of biodegradable and/or bioabsorbable material in the range of about 20 to about 500 nanometers and, more preferably, between about 200 and about 1500 nanometers. In another embodiment the delivery system contains particles or nanofibers in irregular shapes to create a non-woven matrix. For purposes of delivery, the implant of the present invention provides an optimal drug concentration gradient of the therapeutic active agent to the local regions of interest. The homogeneity of the drug distribution within the drug delivery system will lost as the delivery system is degraded allowing the release of the therapeutically active agent. In at least one aspect, the implant of the present invention may further comprise an insertion cannula for delivery of the therapeutic agent to the subject.

In one exemplary embodiment of the present invention, the amorphous form of Polylactic acid/Polyglycolic acid copolymer (PLGA or PLAGA) was chosen as the device material. This biodegradable synthetic polymer has a Molecular Weight of 40-100 kDa and is fully decomposed in 60-90 days (observed in scaffolds for rat stem cells). Middleton et al., (March 1998), Medical Plastics and Biomaterials Magazine, 30.

All PLGA polymers have low polydispersity index ratios (P.D.I.) due to ring-opening polymerization. For example, the P.D.I. ratio for PLA is around 1.6-1.9 in order to maintain mechanical strength and structural consistency. A more amorphous form of the polymer can be used for drug delivery devices while the crystalline form is good for building scaffolds and other sturdy biodegradable structures. However, the relatively high temperatures used in manufacturing these polymers prohibit the use of certain drugs (often. Tₘ lower than manufacturing temperature) and controlling the release of drug agents can be difficult.

Ring-opening polymerization is the most common method of commercial production of PLA and PGA with an insertion mechanism using a metal oxide catalyst. Source: Fried, (1995). Polymer Science and Technology (2^{nd} ed.). Prentice Hall. Ring-opening polymerization yields high-molecular-weight materials, with approximately 1-3% residual monomer present and dimerization of glycolic acid results in glycolide monomers. PGA is the simplest linear aliphatic polyester with a melting point of 220-225° C. and a glass-transition temperature (T_{g}) of 35-40° C. due to its high crystallinity (45-55% crystalline). The higher the crystallinity in an amorphous material such as PGA, the higher the T_{g}, or temperature at which the polymer chain becomes weak and allows for molecular mobility. Such high crystallinity prevents solubility in most organic solvents with the exception of highly fluorinated organics such as 1,1,1,3,3,3-Hexafluoro-2-propanol (HFIP). In vitro experiments have shown that enzymes, buffer (moisture), pH, annealing treatments, and gamma radiation enhance degradation of PGA. For, such reasons at least one aspect of the present invention employs low humidity ethylene oxide gas sterilization procedures and moisture-proof packaging.

In another embodiment of the present invention, PLA (poly-lactic acid) is prepared from the cyclic diester of lactic acid (lactide) by ring opening polymerization. Lactic acid exists as two optical isomers or enantiomers (D and L), the L-enantiomer (semicrystalline) occurs in nature, and a D, L racemic mixture (D,L-PLA, an amorphous polymer) can be made by synthetic preparation of lactic acid.

Poly-L-lactide (L-PLA) is about 37% crystalline with a melting point of 175-178° C. and a T_{g} of 60-65° C. (See, www.birminghampolymers.com/biodegradation.htm.)

Poly L-lactide is more resistant to hydrolytic degradation than the amorphous DL form, due to its higher crystallinity. Semicrystalline PLA exhibits high tensile strength and low elongation, resulting in a high modulus (more suitable for load bearing devices). D,L-PLA is an amorphous polymer exhibiting a random distribution of both isomeric forms of lactic acid, is unable to arrange into an organized crystalline structure, and has lower tensile strength, higher elongation, and faster degradation time (ideal for drug delivery systems).

The time required for L-PLA implants to degrade depends on polymer quality, processing conditions, implant site, and physical dimensions of the implant. (See, www.courses.ahc.umn.edu/medical-school/BMEn/5001/notes/bioabs.html. In vivo studies in rats showed an absorption time of about 1.5 years for 50-90 mg samples of radiolabelled D,L-PLA implanted in the abdominal wall. Id.

In radiolabelled implants, metabolism of the polylactide resulted in excretion primarily via respiration (CO₂) and exposure to gamma radiation showed a decrease in molecular weight. The degradation time of L-PLA is more than two years for complete absorption, much slower than D,L-PLA.

Amorphous copolymers have a compositional range between 25-70 mole percent glycolide, where pure polyglycolide is about 50% crystalline and pure poly-L-lactide is about 37% crystalline. A copolymer of 50 mole percent glycolide and 50 mole percent D,L-lactide degrades faster (in 50-60 days) than either polymer independently, or copolymer with different percentages of either polymer. A copolymer of 90 mole percent glycolide and 10 mole percent L-lactide was developed by Ethicon Inc. under the trade name Vicryl®, which absorbs within three to four months but has a slightly longer strength retention time. (See, birminghampolymers.com/biodegradation.htm.

In general, the 65:35, 75:25, and 85:15 D, L-lactide/glycolides have progressively longer lifetimes in vivo, with the 85:15 lasting about five to six months in vivo. Poly (D, L-lactide) requires about 12-16 months to biodegrade completely, and poly (L-lactide), being more crystalline and less hydrophilic, can take 1.5 to 2 years in vivo to degrade. (See, www.alkermes.com/polymer/products.htm.

Degradation occurs via hydrolysis, by breaking down the D,L-PLGA copolymer into D,L-PLA and PGA. Longer exposure time to water and metabolic processes further reduce the polymer to lactic and glycolic acid respectively. These are excreted from the body as waste byproducts through carbon dioxide and urine.

Biodegradable systems manipulate natural biological processes to degrade the polymer, therefore releasing the drug that was trapped in or on the polymer. In such systems, the chemical structure of the system changes and the release rate is often governed by the degradation rate. Most biodegradable polymers are designed to degrade via the hydrolysis of the unstable polymer chains as soon as they come into contact with water. In the simplest mechanism of hydrolysis, water penetrates the bulk, preferentially attacking the chemical bonds in the amorphous phase and converting long polymer chains into shorter chains which are water-soluble and biologically compatible.

For some polymer materials such as polylactides, polyglycolides, and their copolymers, the polymers will break down to lactic acid and glycolic acid, enter the Krebs cycle, and be further broken down into carbon dioxide and water and excreted out of the body by normal biological processes. Since degradation initially occurs in the amorphous phase, depending on the extent of crystallinity, the reduction in molecular weight does not affect physical properties or functionality of the device. One embodiment of the present invention is held together by the crystalline regions and remains intact until the degradation reaches a critical point, when the entire device collapses.

Homopolymers (polyglycolic acid and polylactic acid) are crystalline, densely packed, and able to hinder hydrolytic attack, unlike random copolymers which are amorphous. For amorphous polymers, a hydrophobic layer on the surface can be generated, forming an absorbable, adherent, non-flaking lubricant which repels water (molecules cannot reach chain segments in amorphous areas to initiate hydrolysis), slows absorption, and improves the retention of tensile strength. (See, birminghampolymers.com/biodegradation.htm.)

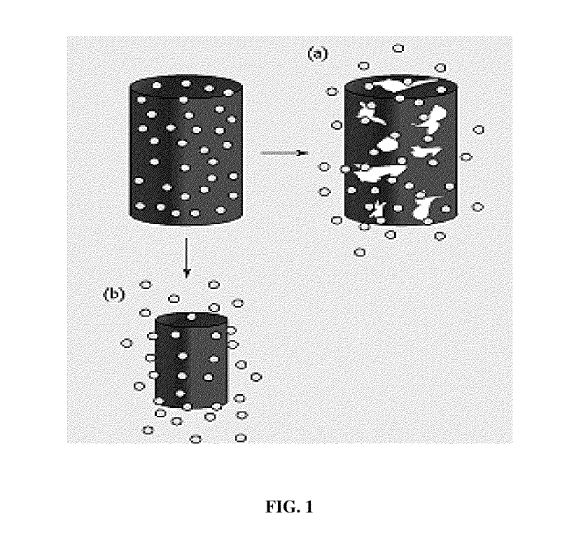

Through bulk erosion, the polymer degrades in a fairly uniform manner throughout the matrix, or by surface degradation as in such materials as polyanhydrides and polyorthoesters. In surface degradation, the release rate is proportional to the surface area of the drug delivery system, resulting in a slower rate of conversion of the polymer into water-soluble materials. A hydrophobic polymer whose chemical bonds are highly susceptible to hydrolysis can experience a form of surface degradation, but is often referred to as bioerosion. See Middleton et al., supra. FIG. 1(a) illustrates bulk erosion and FIG. 1.(b) shows surface degradation in general.

However, it can be appreciated by those of ordinary skill in the art that bulk erosion causes a somewhat inconsistent release of drug. The initial rate of release can vary depending on the amount of drug loaded into the polymer, and its hydrophilic or hydrophobic properties (controlled delivery is directly dependent upon the nature of the polymer). The factors that affect release rate are chemical structure, chemical composition, processing conditions, morphology, and site of implantation. Chemical structure includes molecular weight distribution, shape, and physical factors (shape and size changes, variations of diffusion coefficients, mechanical stresses, and stress- and solvent-induced cracking). Chemical composition involves the presence of ionic groups, molecular weight, the presence of low-molecular weight compounds, adsorbed and absorbed compounds (water, lipids, ions), physicochemical factors (ion exchange, ionic strength, pH), and mechanisms of hydrolysis (enzymatic versus aqueous). Brannon-Peppas, (1997), Medical Plastics and Biomaterials Magazine, 34.

Processing conditions with storage history, sterilization processes, and annealing have an affect on the final overall functionality of the release system. Distribution of repeat units in multimers, the presence of unexpected units or chain defects, and configuration structure, all fall under the category of morphology (amorphous/semicrystalline, microstructures, residual stresses). Brannon-Peppas et al, supra.

The present invention employs polymers purchased from Lakeshore™ Biomaterials, which are used in exemplary embodiments of the present invention. Such polymers are biodegradable polyesters that undergo resorption, breakdown and/or assimilation of physiological conditions at varying rates depending on chemical characteristics of the polymer and attributes of the device. The preferred polymers are degraded via hydrolysis of ester linkages and would ultimate continue until gradual erosion of the delivery system, implant or the incorporated device. Like their general counterparts, the final products of PLGA degradation are lactic acid and glycolic acid, water soluble, non-toxic products of normal metabolism, that are either or further metabolized to carbon dioxide and water.

One embodiment of the present invention utilizes a D,L-polylactic-polyglycolic (D,L-PLGA) electrospun polymer fiber delivery system with commonly used active agents to simulate drug release. In this exemplary embodiment, creating such a system by the electrospinning method, drug delivery occurs by direct implantation of the spun mats after excision of the tumor, thereby decreasing secondary toxicity of the drug. The drug delivery mats of at least one embodiment of the present invention may be compared with the Gliadel® wafer in both size of device and method of implantation. The drug delivery system of the present invention release the content of its drug by hydrolysis of the polymer fibers at the target site, thus providing high concentrations of drug to the tumor bed while greatly limiting any systemic side effects. The present invention is not limited to the instantly disclosed electrospun drug mats, rather the present examples are merely for purposes of demonstrating at least one working example as well as the unexpected advantages of electrospinning in drug delivery within the scope of the present invention.

The DLPLGA (D-L-Poly-Lactic-Glycolic-Acid) polymer for the present invention was obtained from Lakeshore™ Biomaterials in lactide/glycolide ratios of 85/15, 75/25, and 50/50 mole percent. Due to their highly hydrophilic nature, the polymers were kept in airtight bags in the freezer (5° C.). The samples of 85/15, 75/25, and 50/50 DLPLGA are all FDA and Good Manufacturing Practice (GMP) approved.

The molecular weight of the 75/25 DLPLGA was 113 kDa, 123.6 kDa for the 85/15 sample, and 84.8 kDa for the 50/50 DLPLGA sample. The molecular weight was important in determining how easily the polymer would dissolve in the solvents and an estimate on how spinnable the samples would be when used with the electrospinning method.

The preferred solvents to be used in the present invention are organic solvents such as an alcohol, dicholoromethane, chloroform or the like. In at least one embodiment, dicholoro-methane and chloroform were used to dissolve both the DLPLGA and the active agent without disrupting the polymer chain or functionality of the drug. Both solvents and 1,1,1,3,3,3-hexafluoro-2-propanol were used to spin DLPLGA fibers of various diameters without the drug.

The two model active agents used in certain exemplary embodiments of the present invention were Guaifenesin and Beta-Estradiol. The target release and chemical properties of these active agents are similar to those of the commercial product Gliadel®. Gliadel® combines a biodegradable polyanhydride copolymer (p(CPP:SA) 20:80, 192.3 mg per wafer) with BCNU (7.7 mg per wafer or 3.85% wt/wt) by means of compression molding. The selected model drugs have properties that span the behavior of a variety of drugs. Observed degradation and release behavior of these two very different model drugs provides a rationale for this drug delivery device to be used in combination with other drugs for other health issues. Those of ordinary skill in the art can appreciate that the electrospinning method could be combined with any number of additives and polymers to create drug delivery devices. Although chemically all active agents are different, the presently disclosed models provided insight to the drug delivery system seen in exemplary embodiments of the present invention.

The present inventors after searching through the entire drug database, identified that only these two drugs come close to the molecular weight of BCNU. Since chemotherapeutic agents have complex chemical structures that are unique to their functionality, the present inventors believe their model adequately represents the behavior of a BCNU delivery system.

These two model drug choices compared to BCNU do have similar MW and are small molecule solid drugs. The solubility of chemotherapeutic agents, particularly BCNU is not a well researched area due to the lack of need for that information. Chemotherapeutic agents have the ability to cross the blood brain barrier due to a combination of solubility, chemical structure, size, and some unknown chemical phenomena. Due to their strong effects to fight the progression of cancer cells, they have been approved for use by the FDA and researched to maximize release in drug devices or combination therapy.

### EXAMPLES

(1-15) wt % solutions were made by dissolving polymer (and drug) in solvent (DCM, TCM, or HFIP). The electrospinning process used in certain exemplary embodiments of the present invention is shown as follows:
1) The syringe of polymer solution attached to a 16-26 gauge diameter metal capillary was prepared for the syringe pump.
2) The needle was placed horizontally and perpendicularly to the vertical surface at set distances to collect the electrospun nanofibers.
3) The electrode wire was connected to the syringe needle, with the vertical surface grounded.
4) Once the flow of the solution was constant, the power supply was turned on and the voltage set to the desired power output.

The electrospinning device utilized in multiple exemplary embodiments of the present invention was designed and constructed in the Medical Device and Concept Lab at the New Jersey Institute of Technology (NJIT) by S. Shanmugasundarum.

The voltage source, Glassman model ps/fx40p07.5ge9, of 0 to +35 kV was used to induce charge in the polymer solution. The metal capillaries were bought from Fisher Scientific, in sizes 22-18 gauge. The 20 gauge capillary had an outer diameter of 0.914 mm and inner diameter of 0.584 mm. The 18 gauge had an outer diameter of 1.27 mm and inner diameter of 0.838 mm. The collecting surface consisted of multiple materials, most of which were obtained from the local grocery store or hardware store.

Certain exemplary embodiments of the present invention were cut to create discs with a diameter of 12.7 mm to match the diameter of the Gliadel® wafer. Although embodiments of this invention have these specific physical properties there is nothing to indicate changing the size for alternative embodiments. Degradation profiles of 85/15, 75/25, and 50/50 DLPLGA discs (micron sized fibers) were determined by incubation in 13 mls of PBS (pH of 7.41) at 37° C. for a total of three months. Degradation profiles of 50/50 DLPLGA discs with nano sized fibers and beaded fibers (inconsistencies in fiber morphology) were determined by incubation in PBS at 37° C. for a total of two months (this was based upon the results of the micron sized incubation). All of the above incubations were performed under stagnant solutions and samples were extracted at scheduled times during each month for analysis.

Degradation and release profiles of 1 wt % Guaifenesin, 3 wt % Guaifenesin, and 1 wt % Beta-Estradiol were obtained by incubation in 13 mls of PBS at 37° C. for two months. Incubation was performed in both stagnant and dynamic solutions, with samples extracted at scheduled times during each month for analysis. A mini study was conducted for 5 wt % Guaifenesin, incubated in PBS at 37° C. for ten days under stagnant solution to understand drug loading affects on the release and degradation profile. A second mini study was conducted combining both drugs into one mat, using 1 wt % of each drug. These mats or implants were incubated in PBS at 37° C. for ten days under both stagnant and dynamic solutions to determine if combinatorial drug loading by the electrospinning method was possible and what type of release would be observed.

During testing embodiments of the present invention were characterized in part in the following ways:
Differential Scanning calorimetry (DSC-TAQ100) was used for thermal analysis by measuring molecular mobility and thermal transitions through heat flow (can either be a heat/cool/heat process or a heat process at a heat rate of 10° C. and cool rate of 5° C.).
Thermal Gravimetry Analysis (TGA-TAQ50) was used to measure loss of weight as a function of temperature. Samples were run under both nitrogen and oxygen.
Scanning Electron Microscopy (SEM) uses an electron microscope (model LEO 1530VP) operating with a beam of focused electrons across an object to produce a high-resolution image. All samples were carbon coated and imaged at a magnification of 800× using 1-2 kV with the sample mount 3-6 mm away from the objective. Fiber diameters were measured with J-peg software provided by the National Institute of Health (NIH).
Gel permeation chromatography (GPC) to determine weight average molecular weight (M_{w}) was carried out using a Waters Breeze system equipped with a 717plus autosampler, a 1525 binary HPLC pump, a 2487 dual 1 absorbance detector, and a 2414 refractive index detector. Two styragel columns (Polymer Laboratories; 5 mm Mix-C), which were kept in a column heater at 35° C., were used for separation.
UV-Vis spectrophotometer measured absorbance of active agents to analyze release profile during incubation. A Cary 500Scan UV-Vis-NIR duel cell Spectrophotometer (by Varian) was used to measure absorbance between 175-300 nm.
Percent water absorption was measured by weighing samples (all samples weighed together) after test tube extraction by using the following formula: Percent water absorption={[(total wt/total sample #)-drywt]/drywt}*100, where drywt=dry weight measured after extraction, rinsing, air drying, and storage in dessicator. Changes in mat thickness during incubation experiments were measured using a Mitutoyo Digimatic caliper.
Percent shrinkage of mat diameter was determined by measuring the mat with a caliper (Mitutoyo) after extraction, rinsing, air drying, and storage in dessicator. The formula used: Percent mat shrinkage=({original mat diameter−[(extracted mat diameters)/number of extracted mats]}/original mat diameter)*100
The pH measurements of the supernatant liquid were taken at every extraction time using the Oaklon hand held pH 11/mV/Temperature/RS 232 meter (Eurtech Instruments).
Interfibrillar spaces* and Surface Area to Volume Ratio (SA/Vol Ratio) was measured using the procedure and formula:

$$Interfibrillar spaces = [ 1 - ( \begin{matrix}calculated density of mat * \\ known material specific volume\end{matrix} ) ] * 100 \%$$

$$Surface Area to Volume Ratio = \frac{\frac{\begin{matrix} measured mass of mat * \\ known material specific volume\end{matrix}}{cross \text{-} sectional area} * cross \text{-} sectional perimeter }{calculated volume of mat}$$

From Barnes et al. (2006), J. Engineer. Fibers and Fabrics, 1(2), 16-29.

For purposes of the present invention IFS is not defines as interfibrillar spaces in their traditional sense. It rather means the spacing between the fibers in the mat or what will be referred to as interfibrillar spacing or void volume.

### RESULTS

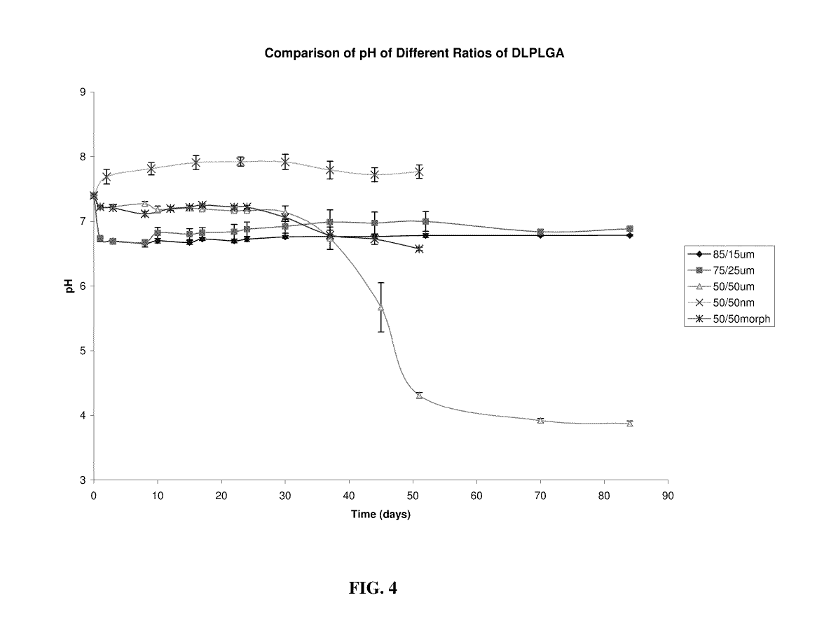

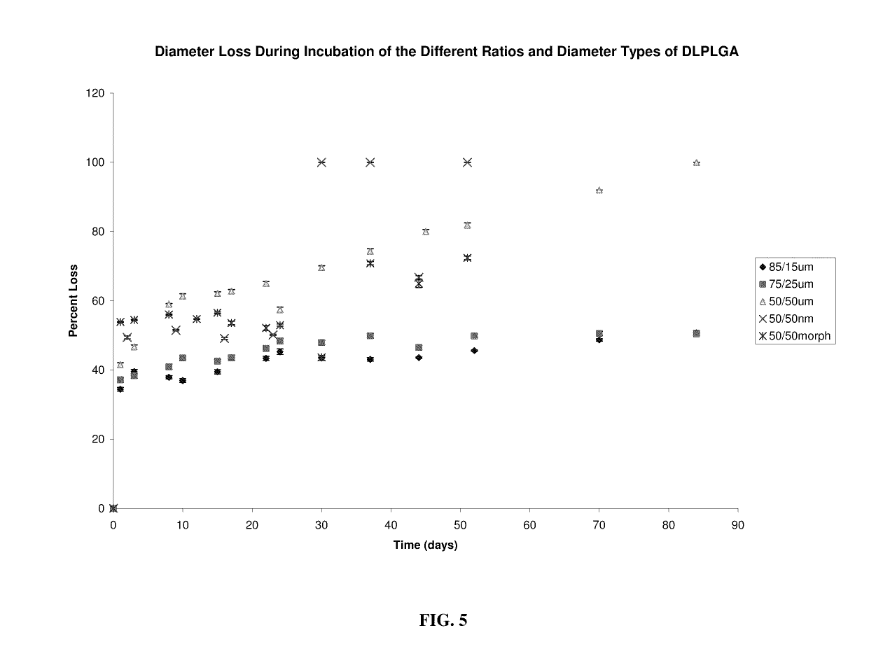

For one exemplary embodiment of the present invention, polymers of three DLPLA-PGA ratios (85/15, 75/25, 50/50) were electrospun to compare degradation profiles of the spun mats to those of the raw polymers (from manufacturer's data). They were analyzed to determine which ratio would serve best in drug delivery for neurosurgical chemotherapeutic applications. Based on the weight average molecular weight (M_{w}) changes and the changes in mat diameter (FIGS. 4,5 respectively), 50/50 DLPLGA was chosen as the polymer to be incorporated with the model drugs by the electrospinning method in one exemplary embodiment of the present invention. 50/50 DLPLGA was electrospun to produce both micron and nanofibers to provide background information on the effects of fiber diameter on degradation (FIGS. 4-5).

The model drugs were chosen based on their molecular weight, physical size and state, and solubility to simulate the behavior of BCNU in the electrospinning process. BCNU would most certainly be an exemplary choice of drug for embodiments of this drug delivery device, however other exemplary embodiments would certainly embrace other drugs.

For one exemplary embodiment, electrospun mats were cut to produce 12.7 mm diameter discs and incubated in stagnant PBS at 37° C. for three months. Three months was chosen to give enough of a population spread for determining the most desired profile for neurological malformations. Stagnant incubation conditions would provide the profile for worst case scenario. Ideally, dynamic fluid would continuously maintain the environmental surroundings, barring any other severe conditions. Degradation can be tracked by change's in pH values due to hydrolytic attack that releases lactic and glycolic acid, therefore lowering the local pH. This local lowering of the pH can occur in areas of the brain where CSF does not flow continuously.

The supernatant pH of samples of this exemplary embodiment of the present invention was tracked to ensure that the pH did not deviate too far from 7.41. If the pH deviates too much above 7.41 or falls too far below, it would be cytotoxic to the glial cells of the brain. Based on FIG. 4, all ratios were able to maintain the target pH for 35 days. The 50/50 nm fiber discs were able to continue this trend for 15 days longer than the micron fiber discs. Surprisingly, the pH of 50/50 beaded discs has comparable behavior. After day 35, the pH of 50/50 μm fiber discs falls drastically to indicate degradation. The 85/15 and the 75/25 discs did not degrade enough to provide a pH change. This is due to the greater ratio of lactic to glycolic acid.

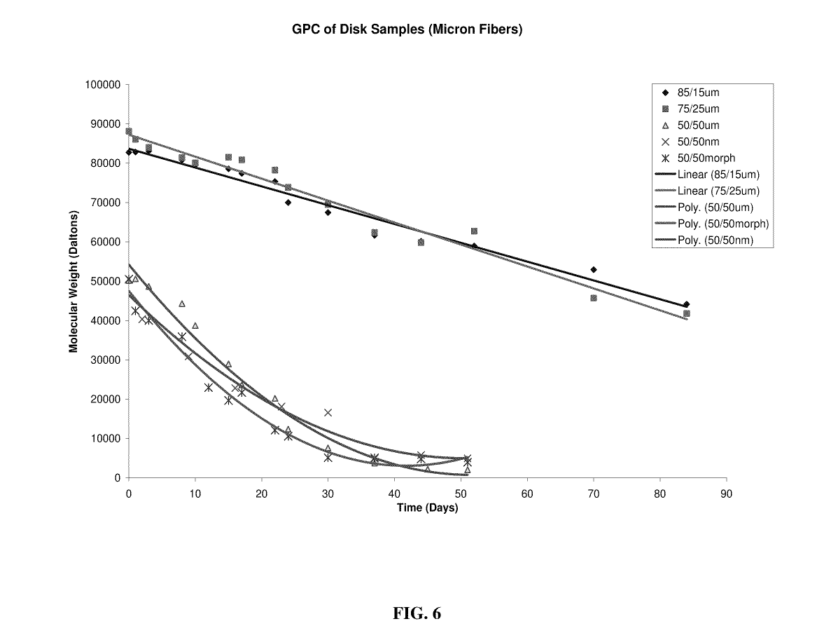

This degradation trend is supported by loss in diameter of the incubated discs (FIG. 5) and the M_{w} changes observed during this time frame (FIG. 6). The loss in diameter of the discs are noted after the discs are extracted from the PBS and dried. The M_{w} changes in the various ratios of DLPLGA confirm complete degradation at day 90 for the 85/15 and 75/25 samples, and day 50 for the 50/50 sample. Studies conducted by Lakeshore™ Biomaterials (the manufacturer of this polymer) of these polymer ratios in pellet form showed the same time frame for complete degradation.

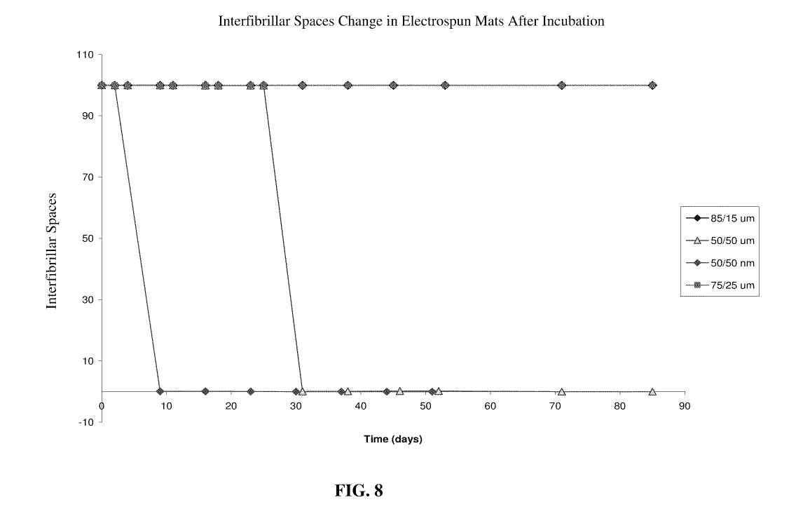

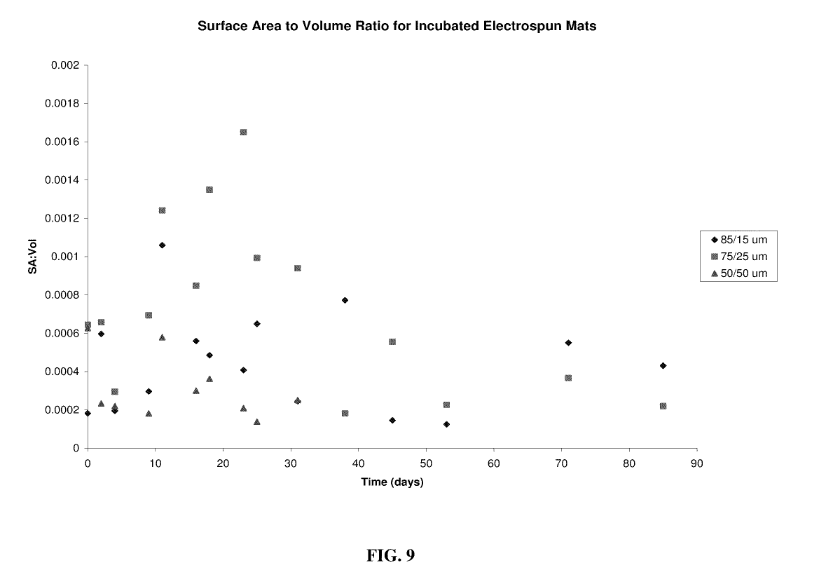

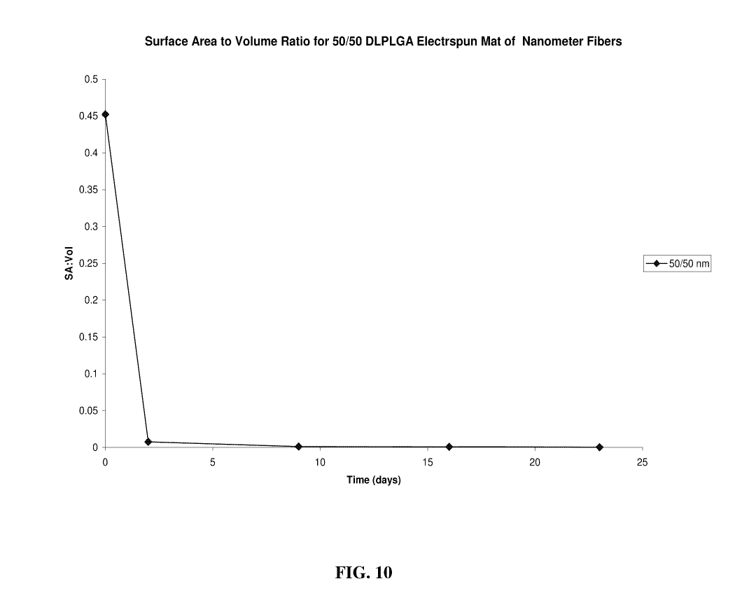

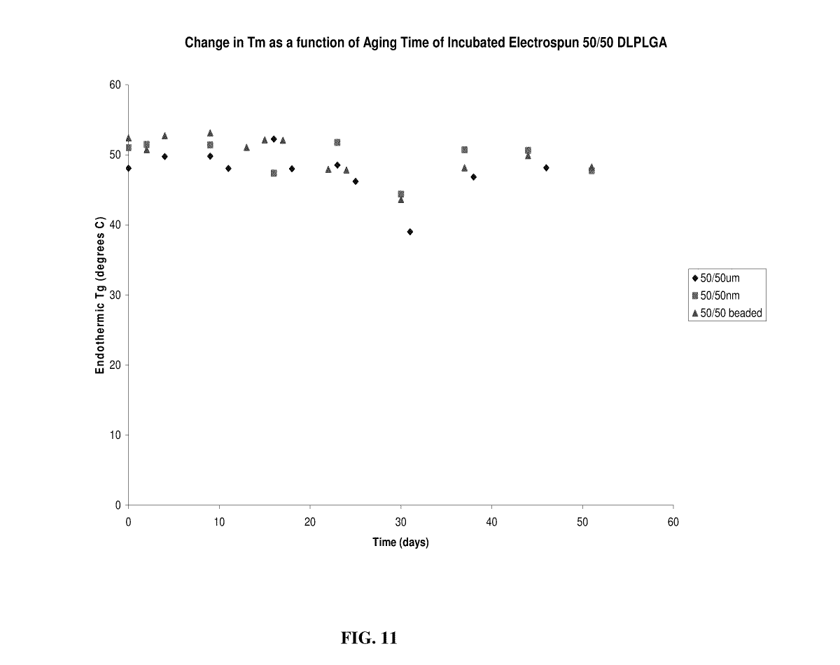

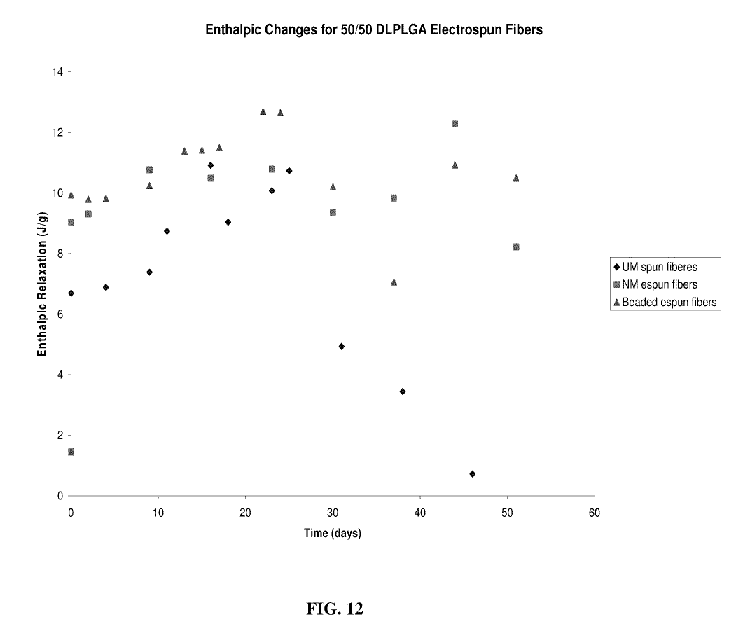

Changes in M_{w} were not due to fiber size or morphology, but to the molar ratio of DLPLA to PGA. Water uptake of all ratios drastically increases after 30 days. Samples of 50/50 nano fiber discs and beaded discs had the greatest uptake. Water uptake is related to the molecular mobility of the polymer chain. The greater water uptake into the polymer, the greater molecular mobility is occurring (amorphous regions followed by crystalline regions), which is, therefore, an indication of enthalpic relaxation. The interfibrillar spaces and surface area to volume (SA:Vol) ratio plots (FIGS. 8-10) support this observation. As would be expected, the smaller diameter fibers would have greater loss in void space in a shorter amount of time. The SA:Vol ratio was a function of both the size of the fibers or beads and the ratio of DLPLA to PGA. It has been shown that as time passes, the polymer aging is represented by the change in the peak of glass transition temperature (T_{g}). This represented by tracking the change in the T_{g} as a function of aging time (FIG. 11) and the enthalpic relaxation as a function of aging time (FIG. 12) of the incubated electrospun polymer discs. Between the raw polymer and the electrospun polymer, it seems as though the electrospinning process may accelerate the aging process of the polymer. This is noted by the difference in the area and sharpness of the peak of the T_{g}. This change in peak appearance could also be due to the exposure of the polymer during prepping to be electrospun and handling of the mats to environmental humidity.

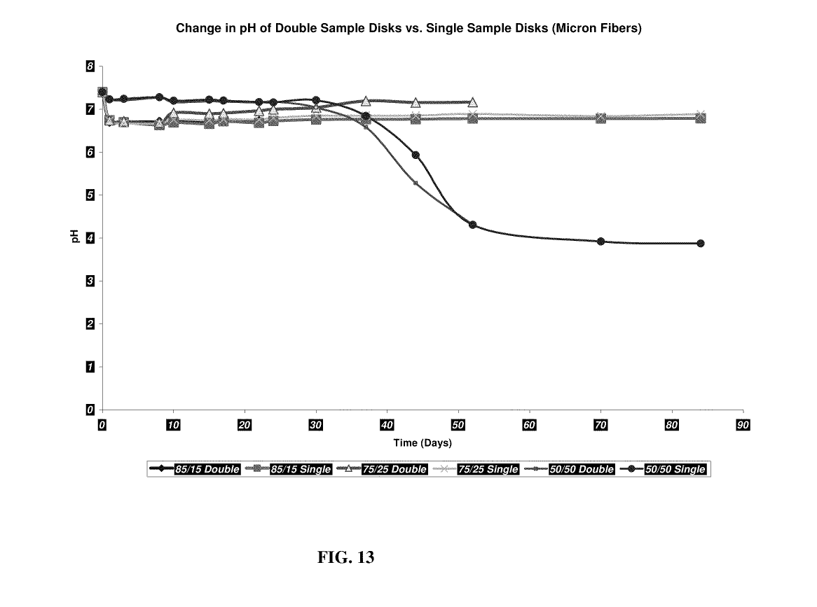

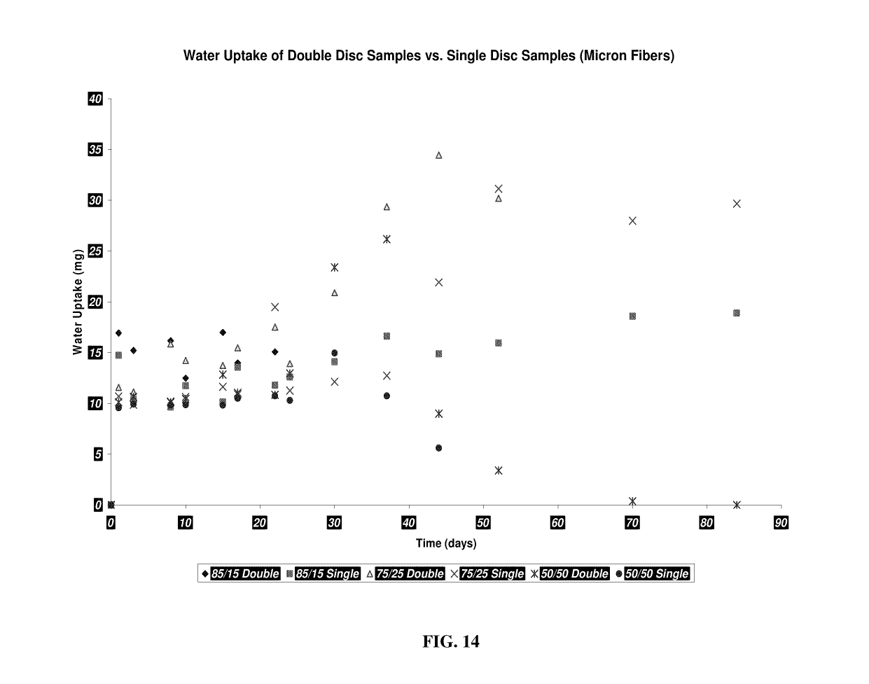

Since embodiments of the present invention are meant to be an implantable device and more than one disc may be needed, a study was conducted to determine the difference between incubating one or two electrospun polymer discs simultaneously. The physical weight and dimension of the one disc would equal that of the two discs. This study was conducted on electrospun micron fiber discs of 85/15, 75/25, and 50/50 ratios of DLPLGA. Based on results from tracking changes in the pH of the supernatant (FIG. 13) and the percent water uptake (FIG. 14), incubating with more than one disc does not affect degradation behavior as long as physical weight and other parameters are kept consistent. This implies that the surface to volume ratio may not be a key parameter.

For this exemplary embodiment, once the degradation profile of the electrospun discs was established, the model drugs Guaifenesin and Beta-Estradiol were co-dissolved in solution and spun via the electrospinning method. The same analytical procedures were utilized to provide information on the drug-polymer interaction. All mats related to this exemplary embodiment of the present invention were spun using the 50/50 DLPLGA and incubated in both stagnant and dynamic PBS at 37° C. Stagnant PBS solution was a test tube solution that was not changed during the entire incubation time. Dynamic PBS solution was a test tube solution that was changed three times a week. Both were used to simulate tumor excision in parts of the brain where CSF does and does not continuously flow. Based on pH changes of 1 wt % Guaifenesin, 3 wt % Guaifenesin, and 1 wt % Beta-Estradiol in both fluid types, the 1 wt % Beta-Estradiol maintained a higher pH than the Guaifenesin. Due to its hydrophobic nature, the beta-estradiol either shielded the polymer from degradation or counter balanced the pH effects of DLPLGA degradation by cationic interactions. The Guaifenesin samples (1 wt % and 3 wt %) maintained lower pH values by anionic interactions, normal for a drug that is more hydrophilic in nature. Both drugs were able to maintain pH values suitable for endothelial cells, and did not increase the rate of degradation. These tests were run for 35 days, just before the 50/50 DLPLGA shows severe signs of degradation (see former graphs).

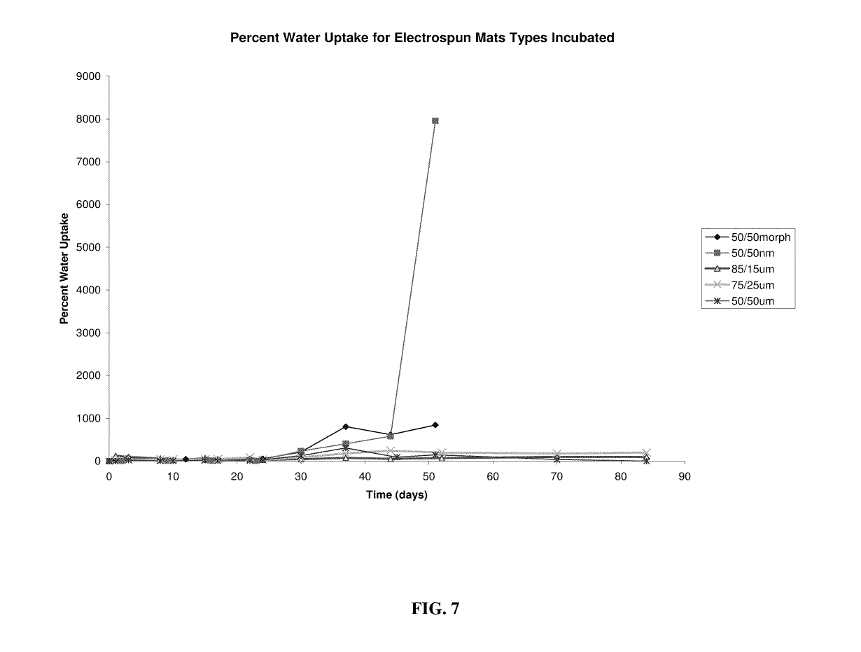

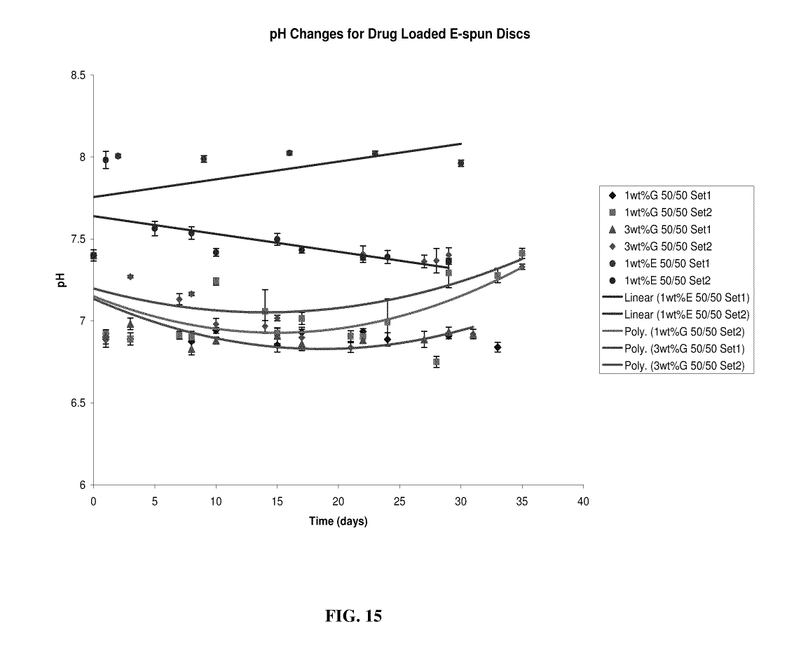

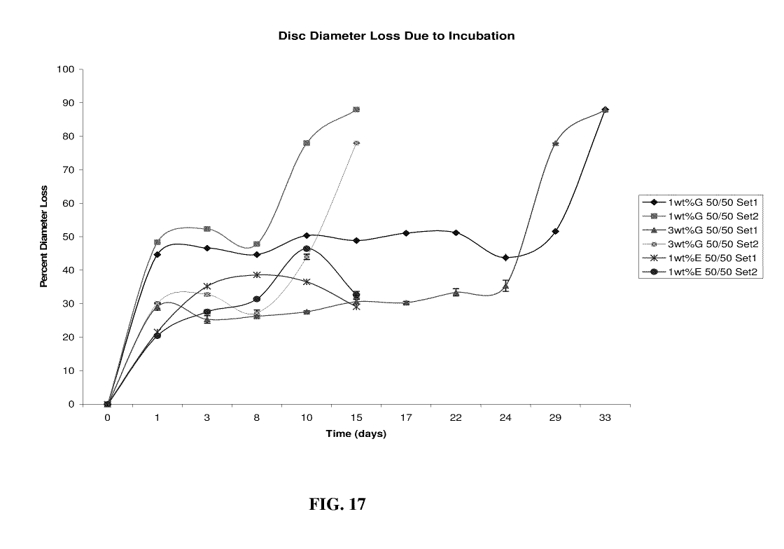

Various embodiments of the present invention show percent water uptake is similar to that of the 50/50 micron, nano, and beaded mats (FIGS. 7 and 15). The 1 wt % and 3 wt % Guaifenesin samples incubated under dynamic conditions had the greatest uptake of water. The fresh solution maintained the process of hydrolysis and prevented equilibrium of particle density in the test tube to be maintained. The 1 wt % beta-Estradiol samples under both dynamic and stagnant conditions had the least water uptake as expected, consistent with the drug's hydrophobic nature. This observation is further supported by the diameter loss of all samples tested (FIG. 15). Disc diameter loss is twice as fast in drug loaded Guaifenesin samples under dynamic solutions than under stagnant and non-drug loaded 50/50 DLPLGA electrospun discs under stagnant conditions (FIG. 17). All drug loaded samples maintained M_{w} changes similar to that of the non-loaded 50/50 DLPLGA electrospun discs. Although based on the pH it seemed as though degradation was not occurring, the rate of M_{w} change confirmed that degradation was indeed occurring.

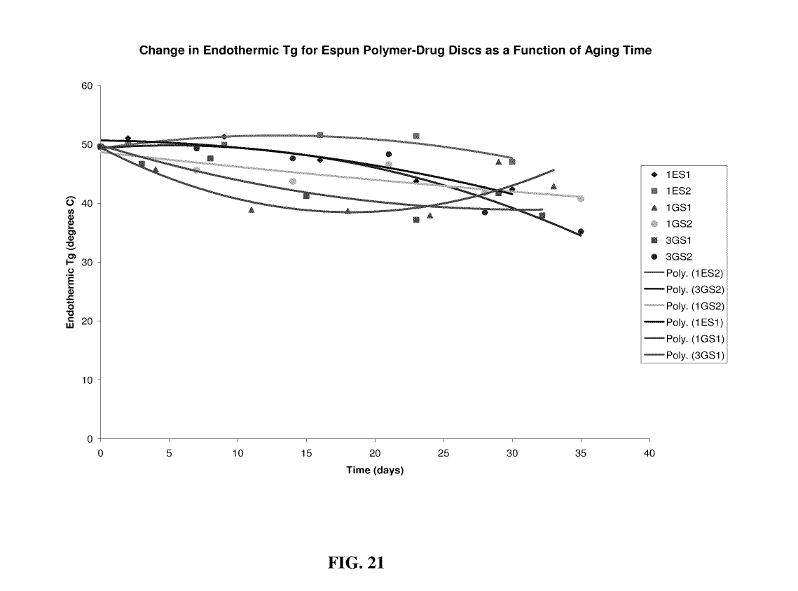

The relationship between change in the interfibrillar spacing and SA:Vol supports the solubility properties of the model drugs. DSC thermograms of the drug loaded polymers during incubation showed the same polymer aging as the non-loaded electrospun 50/50 DLPLGA discs based on the behavior of the T_{g} peak (FIG. 21). The absence of a sharp melting temperature (Tₘ) drug peak at 78.5° C. for 1 wt % Guaifenesin loaded electrospun discs (in both stagnant and dynamic incubation conditions) was due to either lower drug content or drug homogeneity in the disc.

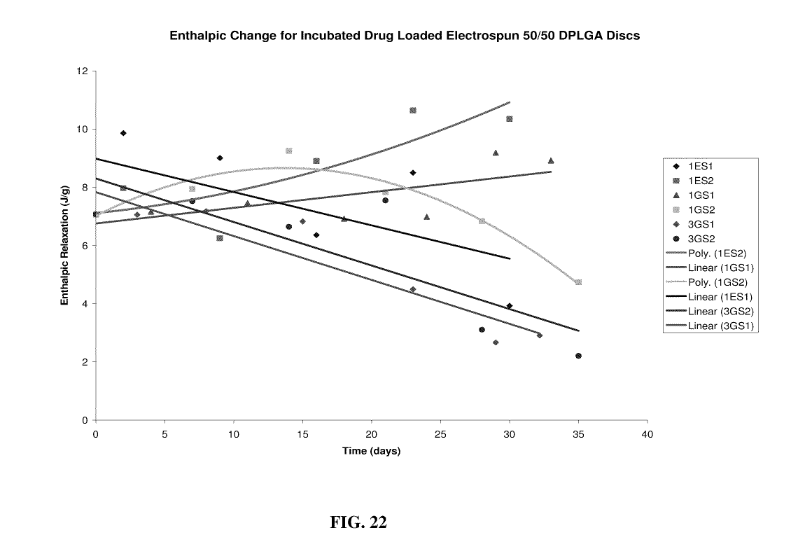

If the drug amount is not large enough, then the signal for the DSC will not be large enough to show on the thermogram. The absence of Tₘ was also seen in 3 wt % Guaifenesin loaded electrospun discs in just the stagnant incubation test. Again, this is due to the particle volume equilibrium of the test tube supernatant liquid that prevented the full release of the drug. This is known because in the dynamic incubation test of 3 wt % Guaifenesin, there is a peak denoting the Tₘ for the drug in week 4 and week 5 of the DSC thermograms. The late presence of the Tₘ peak could be due to re-crystallization of the drug during degradation. The electrostatic forces of the electrospinning method could not overcome the drug properties with time due to the amount of drug loaded. Effects of this re-crystallization is noted in the change in trend of the enthalpic relaxation of the electrospun 3 wt % Guaifenesin (FIG. 22) from that of the other electrospun polymers with and without the drug added. The presence of a Tₘ was seen in 1 wt % Beta-Estradiol loaded electrospun discs, at all sample times for both the stagnant and dynamic incubation tests. The peak broadened as time increased. There was a decrease in the onset of the Tₘ for Beta-Estradiol from 175-177° C. to 120° C., due to the interaction of the polymer with the drug. The affects of the polymer on the drug to cause this decreased onset of Tₘ could be due to the electrostatic forces of the electrospinning method creating this interaction with a drug of low solubility.

Once electrospun, all mats for a particular embodiment of the present invention were placed in a dessicator under vacuum at 373.4 mm Hg and stored in a cool, dark, dry location. When the discs were cut, they were also placed in the dessicator under vacuum at 373.4 mm Hg and stored in a cool, dark, dry location until needed. Some of the drug loaded discs that were utilized in the testing were stored for 7 months to provide information on whether the storage conditions stated were adequate (samples stored with no further degradation occurring). After 7 months, samples were incubated in PBS and blood plasma at 37° C. for ten days. PBS and blood plasma were used to compare the effects of the incubating solutions.

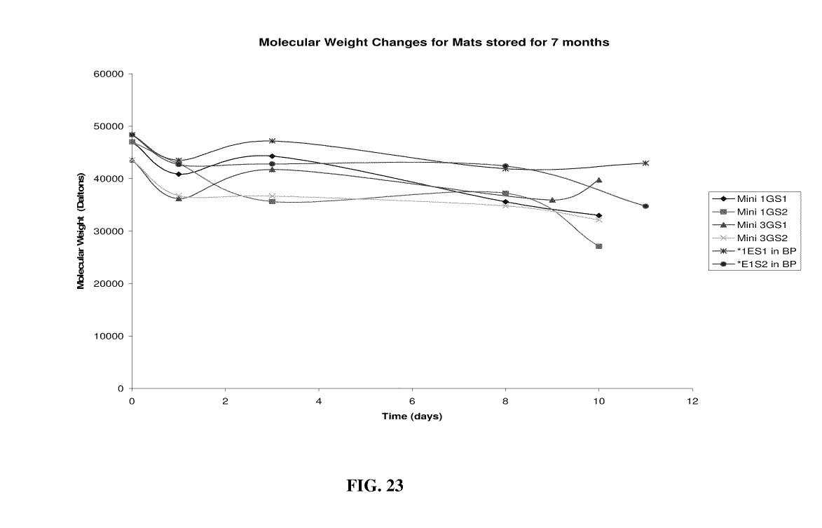

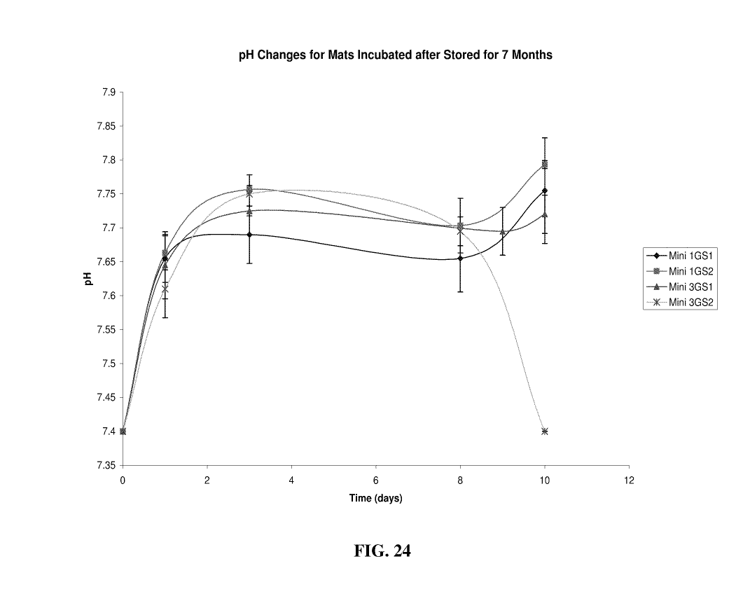

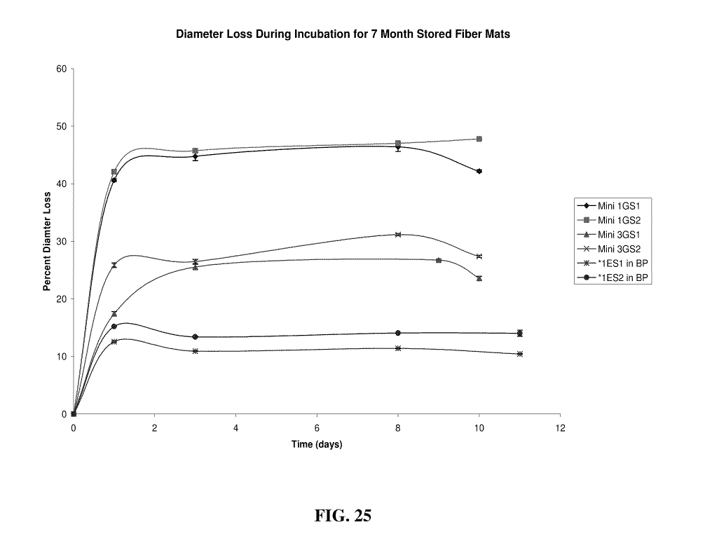

The M_{w} changes of the drug loaded discs stored for 7 months matches the 10 day time frame seen in the initial drug loaded study (FIG. 23). The pH changes of mats stored for 7 months also match the 10 day time frame seen in the initial drug study (FIG. 24). The diameter loss for the Guaifenesin loaded/stored samples follows the same trend as seen in the initial drug loaded study (FIG. 25). The diameter loss for the Beta-Estradiol loaded/stored samples was half of that seen in the initial drug study. This could be due to incubation in blood plasma versus PBS, where coagulating factors are affecting the degradation properties, even though an anticoagulation agent was used. The use of the anticoagulation agent was the reason why pH values could not be ascertained during incubation. This explains the use of histopathological techniques in animal studies to determine inflammatory responses due to the local pH changes in drug device studies. Mittal et al. (2007), J. Control. Rel., 119, 77-85.

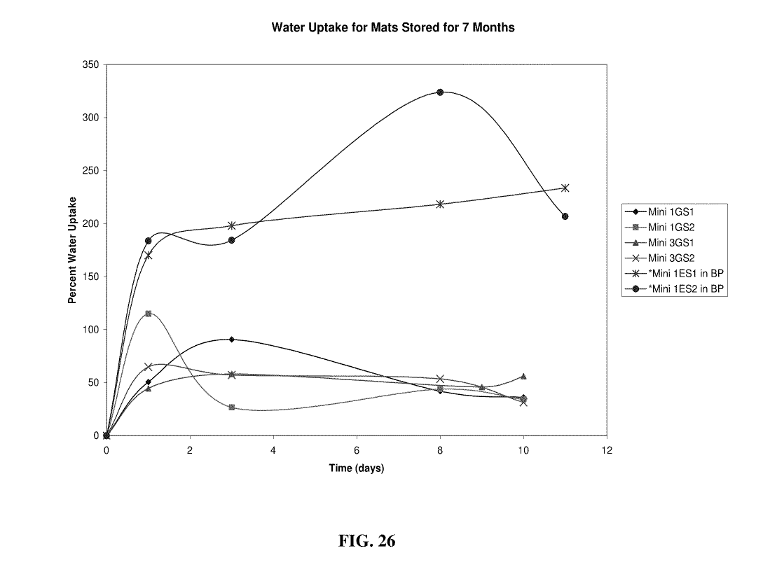

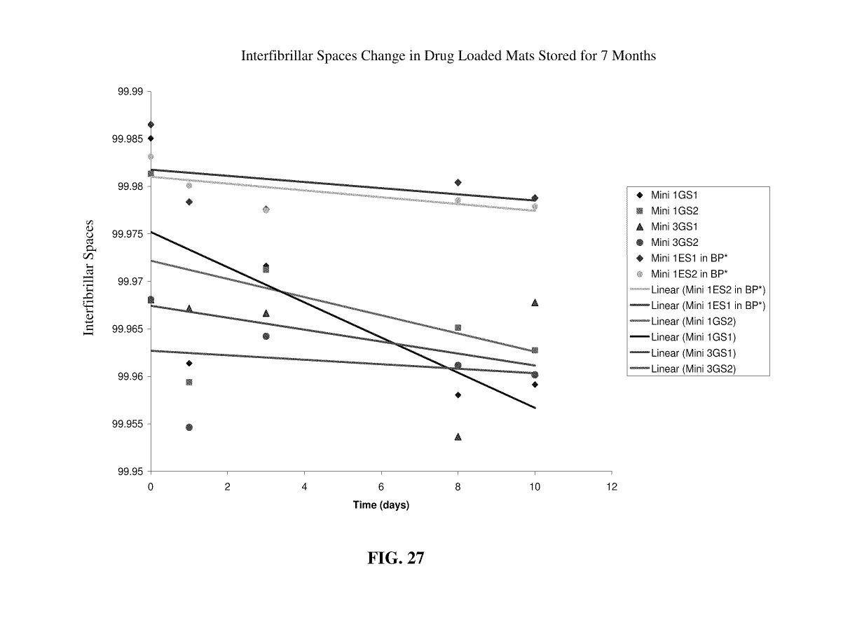

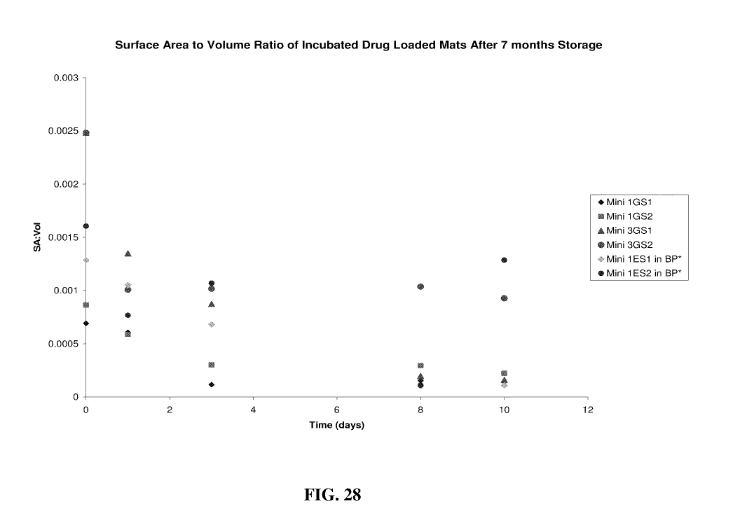

In conjunction, the water uptake of the Beta-Estradiol loaded/stored samples was three times that of the initial drug study. Again this is due to incubation in blood plasma, where coagulating factors could affect the measured weight. For the Guaifenesin loaded/stored samples, the trend is the reverse of the initial drug study (FIG. 26) as related to drug release. The interfibrillar spaces changes (FIG. 27) match exactly the 10 day time frame seen in the initial drug study. The SA:Vol ratio for the drug loaded electrospun discs stored for 7 months are representative of the 10 day time frame from the initial drug study (FIG. 28).

All DSC thermograms for the drug loaded electrospun discs of this particular exemplary embodiment stored for 7 months were exactly matched to those of the initial drug study for 1 wt % Guaifenesin loaded and the 1 wt % Beta-Estradiol electrospun discs (under both stagnant and dynamic testing conditions). The 3 wt % Guaifenesin loaded discs only presented a Tₘ for the drug at day zero. This indicates an acceleration in degradation due to storage, upon incubating the discs after 7 months of storage, that ultimately affects the release of the drug. The re-crystallization of the drug due to the aging of the polymer that was occurring during storage was also observed in the original study. Incubation in blood plasma versus PBS did not have an effect on degradation of the polymer or release of the drug. Visual tracking of these samples through this study was captured in SEM images.

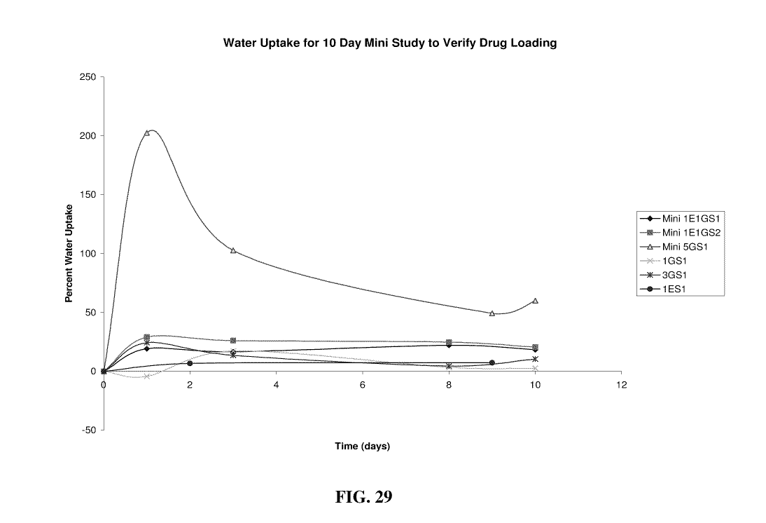

To test the capacity of drug loading for multiple embodiments of the present invention, a mini study (10 day in vitro study) was done with 5 wt % Guaifenesin and a mini drug combination study was done with 1 wt % Guaifenesin and 1 wt % Beta-Estradiol. The maximum amount of drug loaded in the electrospun polymer device was 5 wt % Guaifenesin and 1 wt % Beta-Estradiol. Any larger quantities would cause supersaturation of the solution that would result in precipitation and phase separation of the polymer and drug from the majority of the solvent they were dissolved in. With time, the solvent would evaporate leaving a crystallized non-homogenous mixture of polymer and drug. The 5 wt % Guaifenesin discs had a dramatic increase in water uptake upon initial incubation of the samples in stagnant solution, which eventually leveled off (FIG. 29). Had the discs of this particular embodiment been incubated in dynamic solution, this leveling off may not occur. Comparatively diameter loss, interfibrillar spaces, and SA:Vol ratio for the 5 wt % Guaifenesin samples follows the 1 wt % and 3 wt % loaded samples in the initial drug study.

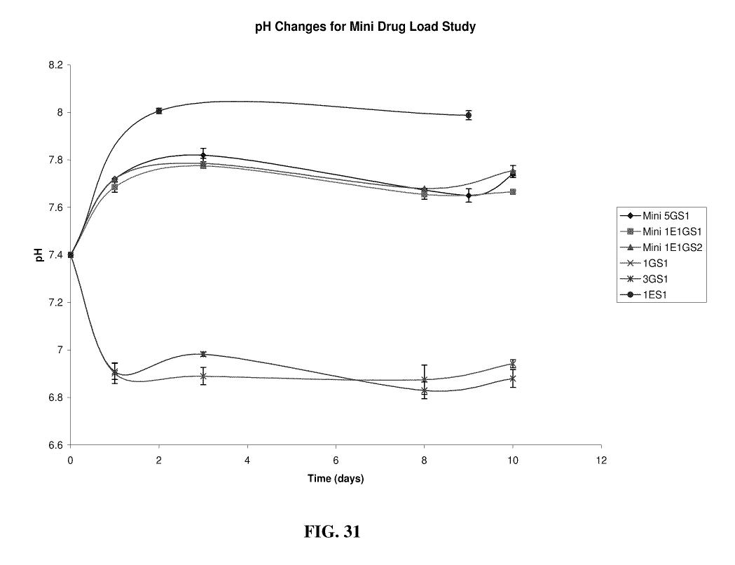

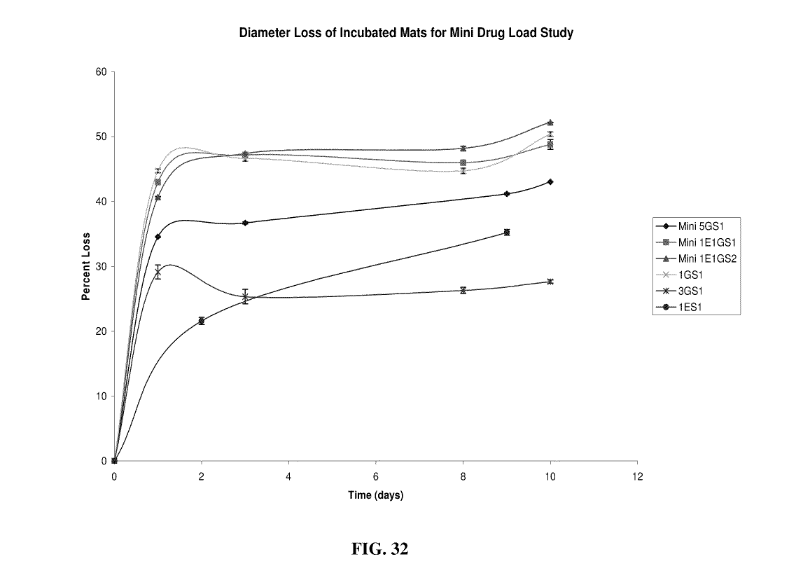

Based on FIG. 31, greater concentration of Guaifenesin in the disc and combination of Guaifenesin with Beta-Estradiol increases the pH slightly above 7.4 (due to solubility and ionic affects). Drug loaded discs of the present embodiment are still below the Beta-Estradiol and above the Guaifenesin pH values noted in the initial drug study. The combination of the model drugs into electrospun discs did not interfere with the polymer properties, but uses the beneficial properties of each drug. The Guaifenesin in these combinatorial discs increased the diameter loss of the disc (due to its hydrophilic nature), similarly to that found in the initial drug study of 1 wt % Guaifenesin (FIGS. 17 and 32).

The DSC thermograms for the 1 wt % Guaifenesin/1 wt % Beta-Estradiol, only showed the Tₘ peak and peak behavior for the Beta-Estradiol and the T_{g} for the polymer. The lack of Tₘ for the 1 wt % Guaifenesin, as was seen in all studies using this concentration of Guaifenesin loaded electrospun discs, follows the same reasoning as in previous studies. Due to the fact that Beta-Estradiol has a higher MW and much lower solubility, would explain the presence of the Tₘ peak and the peak behavior observed in all DSC thermograms. The loading of 5 wt % Guaifenesin produced a sharp Tₘ peak at 74.5° C. for day zero only. The behavior seemed similar to that of the 3 wt % Guaifenesin electrospun discs stored for 7 months. This absence of Tₘ throughout the 10 days of incubation could indicate a burst effect or diffusion release profile that occurred within hours of the first day of incubation. Therefore, release was not be controlled by enthalpic relaxation of the polymer. The electrostatic forces of the electrospinning method can only impact the effect of the polymer on the drug and the modification of the crystalline structure to a critical point based on the quantity of drug loading. However, as seen in the release profiles for this study no burst effect is observed. Instead the drug has extended release of 3.2 mg/day for 10 days. Based on SEM pictures (Appendix C), degradation for 5 wt % Guaifenesin is not visually distinguishable from 1 wt % and 3 wt % Guaifenesin. These SEM pictures show that all drug studies performed on multiple embodiments of the present invention are not on perfectly made electrospun fibrous mats. Therefore, the degradation profile of the beaded 50/50 DLPLGA electrospun mats helped to provide information on the degradation changes occurring during incubation.

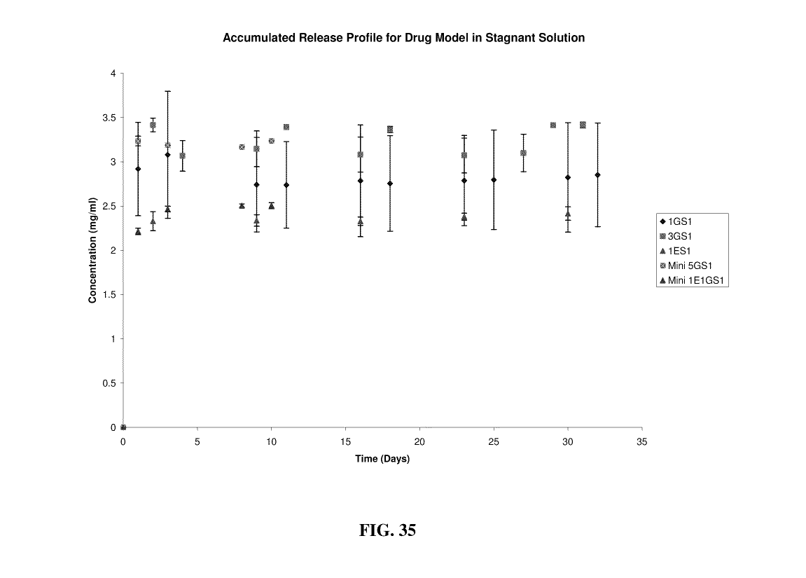

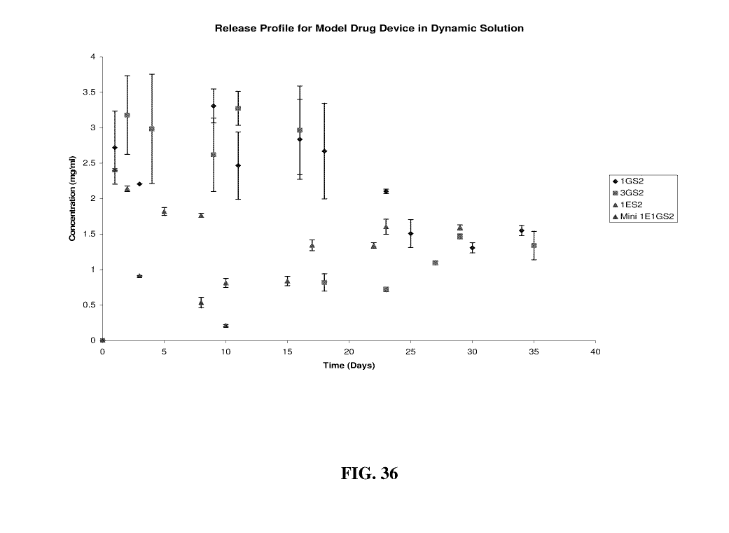

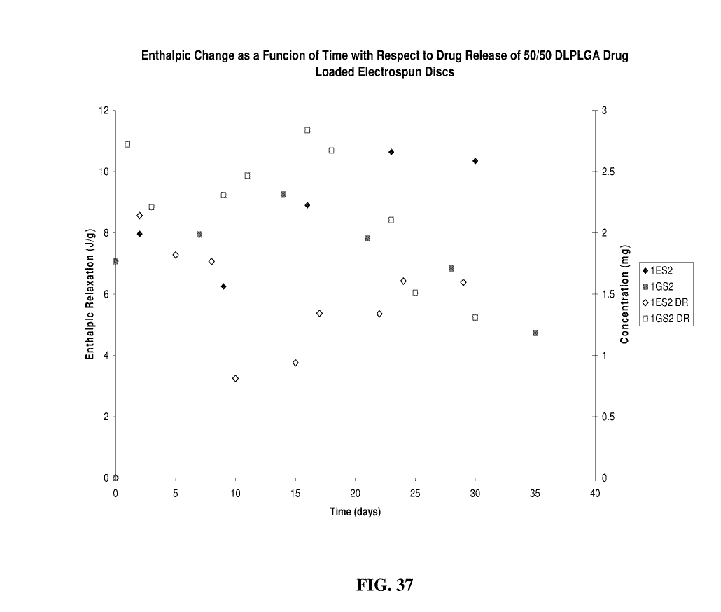

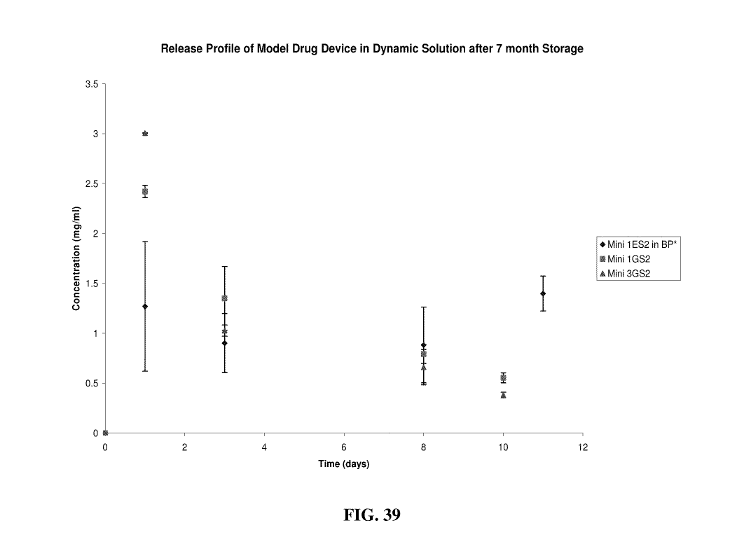

All electrospun drug loaded discs of exemplary embodiments of the present invention involved in this study were analyzed by UV spectroscopy to establish release profiles. Based on the standard curve established, both Guaifenesin and Beta-Estradiol have high absorbance peaks at similar wavelengths. This is verified by other research studies done on these drugs where detection for Guaifenesin ranged between 210-230 nm and detection for Beta-Estradiol between 220-280 nm. All drug discs incubated in stagnant solution showed no accumulated release which would indicate zero order release (FIG. 35). Concentration remained the same from day 1 to day 35. The lack of accumulation exhibited in the stagnant solution studies was due to particle volume equilibrium in the test tube. Looking at the graphs for the dynamic solution incubation, extended release of the drug is shown without exhibiting burst release (FIG. 36). The release rate of the drug was influenced by the enthalpic relaxation of the polymer during degradation (FIG. 37). The rate of release was inversely proportional to the percent water uptake of the electrospun polymer drug discs. The drug concentration released per day is below dosage forms for Guaifenesin (300 mg-2.4 g per day) and exceeds the recommended dosage form for Beta-Estradiol (1 mg per day). These same observations can not be made for the drug loaded electrospun discs stored for 7 months and then incubated in either dynamic PBS or blood plasma. Storage for 7 months in a dessicator under vacuum at 373.4 mm·Hg, in a dark, dry cool location did affect the release profile and the amount released per day. Due to storage conditions, the 1 wt % and 3 wt % Guaifenesin studies exhibited a linear decrease in release over 10 days versus extended release seen in the original study (FIG. 39 and FIG. 36, respectively). This linear decrease in release is directly proportional to the percent water uptake observed for this study. This would explain the trend seen in the DSC thermograms, discussed earlier. Storage conditions also affected the 1 wt % Beta-Estradiol release, by decreasing the concentration released per day. The trend for the storage condition study follows that of the original study between days 20-30 (FIG. 36). The 1 wt % Guaifenesin/1 wt % Beta-Estradiol electrospun disc has the lowest amount of drug released of all samples tested. It also exhibits a burst affect, where both drugs are competing for solubility preference. If the conclusion that the combinatorial drug loaded electrospun disc is equal parts of each drug, and both drugs are released equally, then concentration released is the sum of the two drugs. Therefore, the absorption value measured by UV spectroscopy is that of the two drugs, since they have similar absorbance wavelengths.

The data results of the DLPLGA degradation study of electrospun micron and nano fiber composed mats gives an understanding to the behavior that would be exhibited if these mats were to be used as a drug delivery device such as one of the exemplary embodiments of the present invention. SEM images provided by You et al showed no significant morphological change in the electrospun 50/50 PLGA matrix for the first four days of degradation. You et al. (2005), J. Appl. Polym. Sci., 95, 193-200.

At day eight of incubation, the fibers seemed to be breaking down and partially adhering to each other. After twenty days, the fibrous structure of the PLGA matrix disappeared, and a porous, membrane-like structure, which agglomerated from fragmented chunks, was formed. This same observation is seen only in SEM images of the electrospun fibers of the 50/50 DLPLGA ratio, and not in either the 85/15 or the 75/15. Also, this degradation behavior is exhibited in 50/50 DLPLGA constructs composed of nano fibers, micron fibers, and beaded fibers. You et al explained this degradation behavior by DSC analysis. Since PLGA is an amorphous polymer and has a T_{g} (31° C.) lower than the degradation temperature (37° C.), the thermally induced relaxation of polymer chains could occur during degradation. This would explain the porous, membrane-like structure, not separated by chunks, to be caused by shrinkage due to thermally induced relaxation. You et al. (2005), J. Appl. Polym. Sci., 95, 193-200.

What You et al did not include in their research were DSC thermograms of PLGA during in vitro degradation. Instead they only ran DSC thermograms of PLA and PGA, but not the co-polymer. Although their explanation of thermally induced relaxation might be correct, the DSC thermograms of DLPLGA during in vitro degradation of certain embodiments of the present invention differs. First, the T_{g} of all ratios of DLPLGA used for this research was 43-51° C., supported by product information provided by Lakeshore Biomaterials of the raw polymer. This T_{g} is greater than the degradation temperature of 37° C. that Lou et al claim. The explanation for the thermally induced relaxation could be explained by the hydrolysis process by which the PLGA degrades. Secondly, the endotherms of the DSC graphs of DLPLGA constructs during in vitro degradation did not shift to lower temperatures and the peaks did not broaden. Separately (according to Lou et al), PLA endotherms did not indicate any change and PGA endotherms indicated that preferential hydrolytic degradation occurred with cleavage-induced crystallization in the amorphous regions, followed by further degradation in the crystalline region.

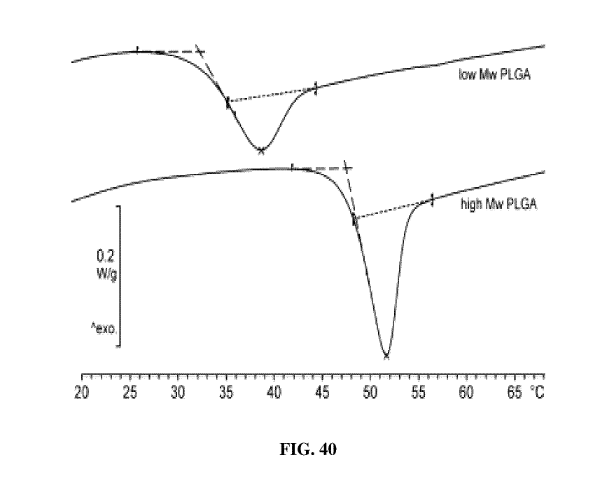

Rouse et al were able to provide information on the determination of the T_{g} for PLGA microspheres (FIG. 40). Rouse, et al. (2007), Int. J. Pharm., 339, 112-120.

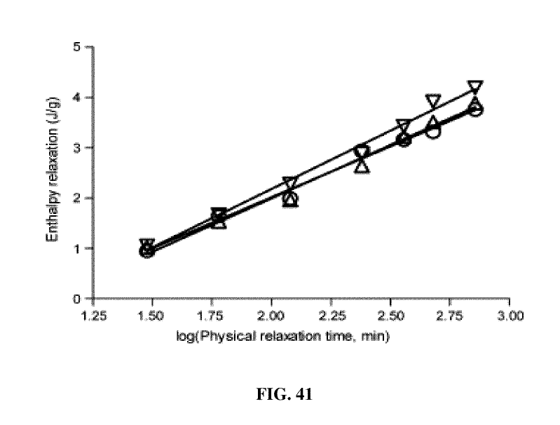

It has been found that a T_{g} for low MW PLGA would be found at lower temperature values (35° C.) than higher Mw PLGA (52° C.). If this is the case, then You et al used a low MW PLGA compared to the high MW PLGA used in this study. Rouse et al incubated PLGA microspheres at temperatures 10° C. below the T_{g} of the high MW PLGA used to observe the accelerated aging process. The increase in T_{g} during physical aging is due to the concomitant decrease in the molecular mobility of the amorphous polymer (crystallization). Evidence that the decrease in molecular mobility is involved in the structural relaxation process was seen by the linear relationship between ΔH and the time of ageing (FIG. 41) provided by Rouse et al. Previously, as shown by Bauwenscrowet and Bauwens (1986) this relationship implied that molecular mobility is a general feature of the structural relaxation of glassy polymers. Source: Rouse, J. J., Mohamed, F., van der Walle, C. F. (2007). Physical ageing and thermal analysis of PLGA microspheres encapsulating protein or DNA. Int. J. Pharm., 339, 112-120.

By using the mathematical model for analysis of the structural relaxation of glassy polymers (Cowie and Ferguson, 1989) Rouse et al found that the mechanism of the structural relaxation involved only the PLGA chains and was limited by the mobility of the PLGA polymer backbone.

Based on the degradation profile of certain exemplary embodiments of the 50/50 DLPLGA electrospun discs and the drug loaded 50/50 DLPLGA discs, the incubation behavior of the polymer only is not affected by the properties of the drugs loaded. However, the release and physicochemical state of the drug was affected by the polymer during processing and under incubation conditions. With respect to the DSC thermograms and structural relaxation, drug properties did not affect the physical aging process. The preparation process (electrospinning) could have modified the crystalline structure of the drug. Absence of a melting peak for the crystalline structure of the drug in DSC thermograms is usually a sign of amorphous or molecularly dispersed drug within the polymer [21, 22]. It can also indicate that the amount of drug is lower than the detection limit of the instrument. Li et al. (1996), J. Cont. Rel., 40, 41-53; Hombreiro-Perez et al. (2003), J. Cont. Rel., 88, 413-428. Bodmeier et al. (1989). Int. J. Pharm., 51, 1-8. Jeong et al. (2002), Bull. Kor. Chem. Soc., 23, 1579-1584.

Drug-polymer interactions (e.g. plasticizing effects of drug on polymer) or polymorph change on the drug can be detected as peak shifts in DSC thermograms. Hirsjärvi et al. (2008), Preparation and Characterization of Poly(Lactic Acid), Nanoparticles for Pharmaceutical Use. Helsinki University Printing House.

This explains why there, is an absence of the Tₘ peak for all experiments conducted with the 1 wt % Guaifenesin loaded electrospun discs and why there is a down shift of the Tₘ peak for all experiments conducted with 1 wt % Beta-Estradiol electrospun discs. For all 1 wt % Beta-Estradiol studies, the downward shift of the Tₘ peak is due to polymorph change on the drug. The drugs used in embodiments of the present invention did not affect the thermo properties of the polymer.

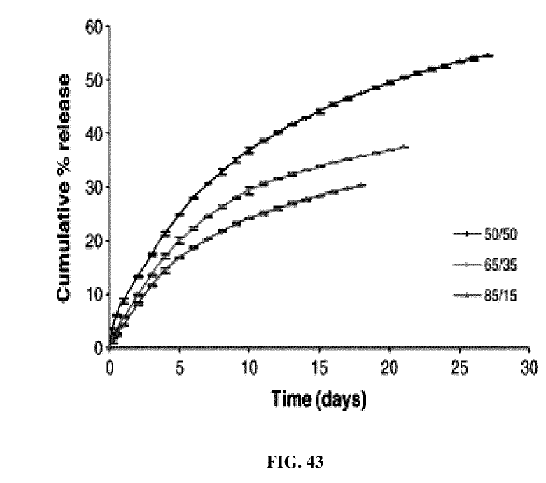

The degradation profile for the drug loaded electrospun fibers indicates a direct inverse relationship between the percent of water uptake and drug concentration found in the supernatant liquid. Therefore, the release profile is directly related to the degradation rate of the polymer, since degradation occurs by hydrolysis. Since the thermograms, with respect to the Tₘ of the polymer do not change when the drug is added to the electrospun matrix, the degradation of the drug loaded discs is subjected to the same structural relaxation. This structural relaxation is the controlling force for the release rate. Based on the trend of structural relaxation as a function of aging time, extended release was shown with the use of the electrospinning method instead of burst or zero order release (denoted by a horizontal or decreasing or increasing vertical trend, FIG. 43). Due to degradation by hydrolysis, and the incubation temperature being far below the Tₘ of the drugs, the drugs could not be degraded. They were released as the polymer degraded.

A non-electrospun control was not used for comparison to an embodiments of the electrospun drug delivery device. A comparison of the electrospun polymer to the electrospun drug loaded polymer of multiple exemplary embodiments provided information on the release profile of both Guaifenesin and Beta-Estradiol. Both were able to produce extended release of the drug and do not exhibit burst effect (considering the duration of the release, FIG. 36), often seen in micron and nano-encapsulation techniques. The drug concentration released per day was approximately 3 mg/ml for both the 1 wt % and 3 wt % Guaifenesin loaded electrospun discs. Since Guaifenesin is meant to be used in the treatment of colds and orally administered, research on prolonging its release has not been necessary. Therefore the release profile can only be compared to the daily recommended dosage of 300 mg-2.4 g per day. The dosage per day exhibited in the results are far below the recommended daily dosage. However, for the type of drug Guaifenesin is, extended release of a consistent daily dosage for weeks is successful. Future research may involve additives to encourage greater release or greater uptake of the drug, or a greater loading of the drug into the polymer.

As for the Beta-Estradiol loaded electrospun polymer discs of multiple exemplary embodiments, release was about two and a half times the daily dosage recommended. This drug had lower solubility and higher MW than Guaifenesin. Compared to other studies done on Beta-Estradiol nanoparticles for oral delivery, electrospun fibers produce a greater concentration of drug released for a greater amount of time without the use of stabilizers (such as PVA and DMAB) and with less drug loading.

These same observations can not be made for the drug loaded electrospun discs stored for 7 months and then incubated in either dynamic PBS or blood plasma. Storage for 7 months in a dessicator under vacuum at 373.4 mm·Hg, in a dark, dry cool location did affect the release profile and the amount released per day. The storage conditions exhibited a linear decrease in release and released half the concentration of drug compared to the initial drug study (upward shift in time from the initial release profile).

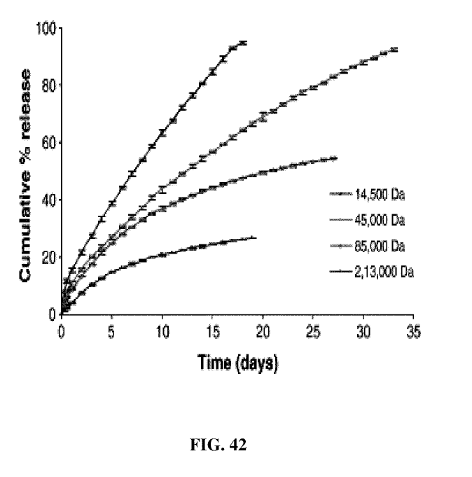

Mittal et al obtained zero order release (PLGA and estradiol (10% w/w of polymer) solution in ethyl acetate) with low molecular weight (14,500 and 45,000 Da) PLGA, while high molecular weight (85,000 and 213,000 Da) and different copolymer compositions followed square root of time (Higuchi's pattern) dependent release (FIGS. 42, 43). Mittal et al. (2007), J. Control. Rel., 119, 77-85.

The bioavailability of estradiol from nanoparticles was assessed in male Sprague Dawley (SD) rats at a dose of 1 mg estradiol/rat. The in vivo performance of the nanoparticles of exemplary embodiments of the present invention was found to be dependent on the particle size, polymer molecular weight and copolymer composition. The Cₘₐₓ of drug in the plasma was dependent on the polymer molecular weight and composition while particle size was found to influence the duration of release, suggesting smaller is better. Source: Mittal, G., et al supra.

Plasma concentration could not be detected past 6 days, whereas in exemplary embodiments of the electrospun drug loaded discs, detection was observed past 30 days in vitro.

The lack of detection in vitro could indicate loss of dosage of the nanoparticles for particular exemplary embodiments of the present invention. The histopathological examination revealed absence of any inflammatory response with the formulations prepared of low/high molecular weight or high lactide content polymers for the studied period. Since electrospun fibers were meant to be an implantable device, the detectable plasma concentration would be higher and for a longer period of time than the oral dosage due to first pass affect. With no observation of any inflammatory response by Mittal et al using the same polymer as in this research, it can be concluded that in vivo studies of electrospun mats would also exhibit a lack of inflammatory response.

The findings of Katti et al were confirmed on the mode of degradation and elimination of polyanhydrides in vivo. Katti et al. (2002), Adv. Drug Del. Rev., 54, 933-961.

Based on p(CPP:SA) 20:80 wafers tested in vivo, the erosion front advances from the exterior to the interior of the wafer in a layer-by-layer process. Initial degradation of the p(CPP:SA) 20:80 polymer wafer occurred more slowly when tested in rat brain than in vitro at pH 7.4. Degradation rates can be varied by co-monomers in the polyanhydride co-polymers and in vivo degradation correlates with in vitro degradation. Source: Katti, D. S., et al. supra.

Since DLPLGA is a co-polymer whose degradation rates can be varied by co-monomers, then it can be concluded that in vivo degradation will correlate with in vitro degradation. The pH data of Gliadel® samples tested showed no deviation from the pH of 7.4 that is necessary for the survivability of endothelial cells. Like the study on Guaifenesin loaded discs and Beta-Estradiol discs, the absence of pH change in the test tube is not an indication that degradation is not occurring. Dang et al reported that the erosion of the Gliadel® wafer in vitro and in vivo is independent of its initial molecular weight. Dang et al. (1996), J. Control. Rel., 42, 83-92.

Erosion of the wafer is characterized by an induction period in which there is a significant decrease in wafer MW, with a small decrease in wafer mass. The release of BCNU from Gliadel® is a combination of the diffusion of drug substance and the erosion of the polymer matrix.

Sipos et al confirmed that despite the more sustained drug delivery achieved with the 50:50 p(CPP:SA) over the 20:80 formula, the in vivo dose-escalation study of efficacy confirmed no difference in survival rates for rats treated with either of the two formulas. Sipos et al. (1997), Cancer Chemother. Pharmacol., 39, 383-389.

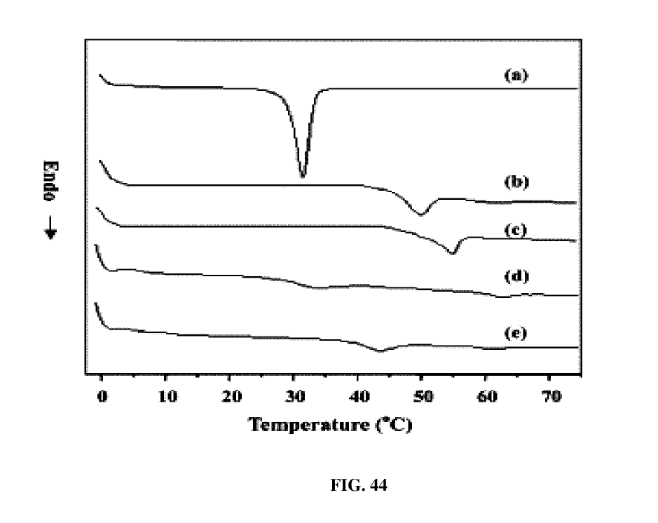

If embodiments of this electrospun drug delivery device using 50/50 DLPLGA was combined with BCNU and the release profile was equal to or greater than that of Gliadel®, it would be a comparable device for neurosurgical chemotherapy. In research produced by Seong et al. BCNU-loaded 75/25 PLGA wafers composed of microparticles were made via the spray drying method. Crystals of BCNU were not detected in BCNU-loaded PLGA microspheres indicating solid-state solution of BCNU and PLGA (FIG. 44). Seong et al. (2003), Int. J. Pharm., 251, 1-12.

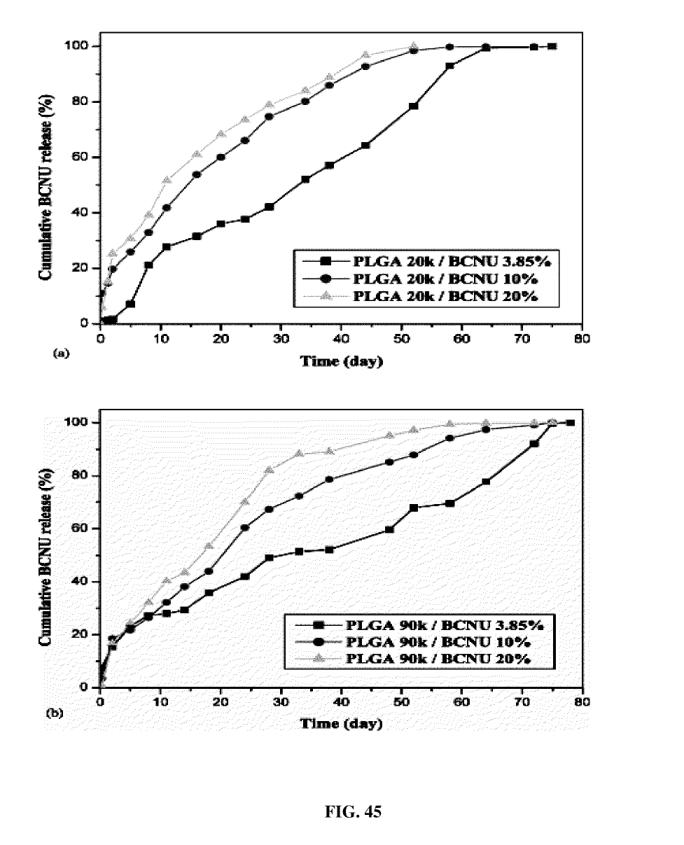

BCNU release rate was near zero-order for up to eight weeks with release rate and release period dependent upon the molecular weight of PLGA, concentration of PLGA, and BCNU loading amount (FIG. 45).

Vogelhuber et al tried to combine BCNU with paclitaxel in both 50/50 PLGA and p(CPP:SA) 20:80 by physical mixing and manual compression. The combination of BCNU with paclitaxel led to simultaneous release according to a time schedule. While the increase in BCNU dosage resulted in more substantial regression of tumor over a period of two months, the combination of BCNU with paclitaxel led to complete remission in some rat brains. Vogelhuber et al. (2002), Int. J. Pharm., 238, 111-121.

By providing a combination study of Guaifenesin and Beta-Estradiol, two drugs of differing hydrolytic characteristics but similar in MW, it can be concluded based on data from Vogelhuber et al that the electrospun drug delivery device would produce similar results.

The combination of 1 wt % Guaifenesin/1 wt % Beta-Estradiol electrospun disc had the lowest amount of drug released of all samples tested, but with burst release. Both drugs were competing for hydrophobic or hydrophilic tendencies. If the assumption that the combinatorial electrospun disc of one particular embodiment of the present invention contains equal parts of each drug, and both drugs are released equally, then the concentration released is the sum of the two drugs. Since the UV absorptions for both drugs were similar, it can be assumed that the absorbance value taken is for both drugs, based on the previous statement. For example, the release of the combination was 1 mg/ml for day 3 of the two or 0.5 mg/ml per day for each drug.

For a hydrolytic process, a water soluble drug is the primary match for a drug delivery device. Verreck et al did the exact opposite research, using electrostatic spinning of a non-biodegradable polymer (polyurethane) with a poorly water soluble drug. Verreck et al. (2003), J. Control. Rel., 92, 349-360.

The release of poorly water soluble drugs was achieved from a non-biodegradable polymer using this type of spinning. The drug release rate can be tailored by changing the drug polymer ratio. That same tailoring could be applied to the present invention.

Xie and Wang were able to electrospun 50/50 PLGA with paclitaxel to obtain fibers of 2.5±0.32 μm and 770±13 nm. Xie et al., (2006), Pharm. Res., 23, 1817-1826.

Again, they experienced difficulty in producing fiber diameters smaller than 770 nm. They were able to show that electrospinning 50/50 PLGA with the help of organic salts (Tetrabutylammonium tetraphenylborate—TATPB) helped reduce the diameter size to 30 nm. These salts were not used to help reduce the diameter of the paclitaxel loaded fibers for the same reasons that this research did not use organic salts to reduce diameter. Precipitation and chemical reactivity was an issue. However, based on the results of the micron encapsulation data of Rouse et al, You et al, and the electrospun 50/50 DLPLGA incubation data for nano, micron, and beaded fibers, nano sized fibers would have a greater release of drug than the micron sized. This is in conjunction with the inverse relationship between amount of water uptake and concentration of drug released, over time. The beaded fibers in comparison to the micron and nano had release profiles which suggest that the fiber morphology may not matter. The size of the morphological pattern (consistency in beading, fiber size, bead size) and the polymer type are the determining factors in rate of release.

Based on the DSC thermograms of the electrospun DLPLGA discs and the drug loaded DLPLGA discs of multiple exemplary embodiments of the present invention, degradation was determined by physical and chemical changes observed. Since the thermograms did not change when the drug was added to the electrospun matrix, the drug properties did not affect the physical aging process.

Storage conditions did affect the aging process, showing visible signs of accelerated physical change and the broadening of the T_{g} peak over a smaller timeframe. The degradation profile for the drug loaded electrospun fibers indicates a direct inverse relationship between the amount of water uptake and drug concentration found in the supernatant. The release profile is directly related to the degradation rate of the polymer, since degradation occurs by hydrolysis.

When compared to the release profile of the Gliadel® wafer, exemplary embodiments of the Guaifenesin and Beta-Estradiol loaded electrospun discs have similar release profiles. The extended release profile of this drug exhibited in this research is a desired trait for neurosurgical chemotherapy. The concentration delivered per day was not equivalent for dosages prescribed for Guaifenesin or a water soluble drug. However, extended release was accomplished for this type of drug. As for Beta-Estradiol, the concentration delivered per day was twice the normal dosage prescribed. As seen with the degradation profiles of the electrospun DLPLGA discs and drug loaded discs tested in this research, tailoring the drug delivery device is possible with the electrospinning method and the loaded wt % of drug. Rate of release is dependent upon the size of the morphological pattern (consistency in irregularities in fiber, fiber size, bead size) and the type of polymer. Fiber constructs are not the driving force behind release, but are suitable for physical parameters needed in how and where the device is implanted. The electrospinning of exemplary embodiments such drug delivery constructs are more beneficial than other devices due to the greater concentration of release for a greater amount of time. The control of drug release by electrostatic forces of the electrospinning method can be tailored to design desired drug delivery.

Malignant malformations are multifaceted, affecting individuals during the most productive years of their lives with devastating consequences. Current treatment carries substantial risk, so developing a system which can safely and effectively treat these lesions is vital. A drug delivery system that is compatible to all grade and type of malformation and is accessible to all areas of the brain is favored. In choosing intracranial tumors as the clinical target problem, embodiments of the delivery device have the ability to have variance in active agent choice and material choice.

Although the systems and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

We claim:

1. A biodegradable resorbable drug delivery system comprising an electrospun bio-degradable resorbable polymeric fiber matrix and at least one therapeutic agent incorporated into the fibers of said fiber matrix, wherein said fiber matrix has an interfibrillar space of at least 65% by volume.

2. The delivery system of claim 1, wherein the interfibrillar space is in the range of 85 to 99.9% by volume.

3. The delivery system of claim 2, wherein said interfibrillar space is greater than 99% by volume.

4. The delivery system of claim 1, wherein the polymer of said polymeric matrix is a polyester selected from the group consisting of poly(alpha- or beta-hydroxyacid) esters and copolymers and mixtures thereof.

5. The delivery system of claim 4, wherein the polymer is a poly (lactic acid), a poly(glycolic acid) or a copolymer thereof.

6. The delivery system of claim 5, wherein said polymer is a poly(lactic-glycolic acid) copolymer, wherein the lactic acid to glycolic acid ratio ranges between about 85:15 and about 50:50.

7. The delivery system of claim 56 wherein the ratio of lactic acid to glycolic acid is about 50:50.

8. The delivery system of claim 1, wherein the therapeutic agent is hydrophobic.

9. The delivery system of claim 1, wherein the therapeutic agent is a chemotherapeutic agent selected from the group consisting of alkylating agents, nitrosoureas, alkylsulfonates, antimetabolites, pyrimidine analogues, purine analogues, antimimotic agents, antibiotics, platinum coordination complexes, aromatase inhibitors, gonadotropin analogs, biological modifiers, or combinations thereof.

10. The delivery system of claim 9, wherein the therapeutic agent is selected from the group consisting of BCNU, doxorubicin, mitomycin, 5-FU, methotrexate, busulfan, cisplatin, hydroxyurea, procarbozine, paclitaxel, docetaxel, vincristine and mercaptopurine.

11. A method of delivering a chemotherapy drug to tissues from which a tumor has been excised in a patient comprising contacting said tissues with the delivery system of claim 9, said delivery system containing an effective amount of said chemotherapeutic agent for delivery to said tissues.

12. The method of claim 11, wherein said tissue from which said tumor was excised is intracranial tissue, said tumor is a glioblastoma and said chemotherapeutic agent is BCNU.

13. The method of claim 11, wherein said interfibrillar space is greater than 99% by volume.

14. The method of claim 11, wherein the polymer of said polymeric matrix is a polyester selected from the group consisting of poly(alpha- or beta-hydroxyacid) esters and copolymers and mixtures thereof.

15. The method of claim 14, wherein the polymer is a poly (lactic acid), a poly(glycolic acid) or a copolymer thereof.

16. The method of claim 15, wherein said polymer is a poly(lactic-glycolic acid) copolymer, wherein the lactic acid to glycolic acid ratio ranges between about 85:15 and about 50:50.

17. The method of claim 11, wherein the therapeutic agent is a chemotherapeutic agent selected from the group consisting of alkylating agents, nitrosoureas, alkylsulfonates, antimetabolites, pyrimidine analogues, purine analogues, antimimotic agents, antibiotics, platinum coordination complexes, aromatase inhibitors, gonadotropin analogs, biological modifiers, or combinations thereof.

18. The delivery system of claim 17, wherein the therapeutic agent is selected from the group consisting of BCNU, doxorubicin, mitomycin, 5-FU, methotrexate, busulfan, cisplatin, hydroxyurea, procarbozine, paclitaxel, docetaxel, vincristine and mercaptopurine.

19. A method for repairing in a patient a blood vessel characterized by a weakened wall, comprising applying to the interior of said weakened blood vessel wall an electro-spun bio-degradable resorbable polymeric fiber matrix having an interfibrillar space of at least 65% by volume.

20. The method of claim 19, wherein said weakened blood vessel is an intracranial aneurysm.

21. The method of claim 19, wherein at least one therapeutic agent is incorporated into the fibers of said fiber matrix, wherein said therapeutic agent is a thrombogenic agent or an agent for reducing blood vessel fragility.
